(19) United States
(12) Patent Application Publication
(10) Pub. No.: US 2025/0195393 A1
(43) Pub. Date: Jun. 19, 2025

(54) COSMETIC COMPOSITION COMPRISING SKIN ACTIVE AGENT

(71) Applicant: L'OREAL, Paris (FR)

(72) Inventors: Ritesh Kumar SINHA, Linden, NJ (US); Hy Si Bui, Piscataway, NJ (US); Xuefei Liu, Edison, NJ (US); Chung-chueh Chang, Clark, NJ (US)

(73) Assignee: L'OREAL, Paris (FR)

(21) Appl. No.: 18/544,254

(22) Filed: Dec. 18, 2023

(51) Int. Cl. A61K 8/81; A61Q 19/00

(52) U.S. Cl. CPC A61K 8/8152; A61Q 19/00; A61K 2800/21; A61K 2800/52; A61K 2800/54; A61K 2800/596

(57) ABSTRACT

A cosmetic composition comprising: (a) a hydrophobic polymer formed as a reaction product of a natural or food-derived oil and a methacrylate or acrylate polymer; (b) one or more solvents capable of solubilizing the hydrophobic polymer; (c) one or more skin active agents; (d) one or more surfactants; and (e) water is described. The composition is an oil-in-water emulsion. Methods from making and using the cosmetic composition is also described.

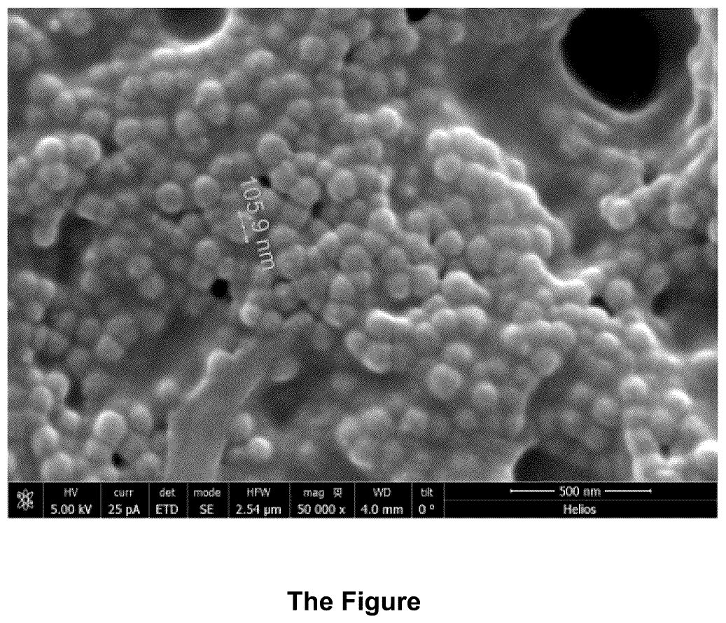

### FIELD OF THE DISCLOSURE

The present disclosure relates to cosmetic compositions in the form of oil-in-water emulsions comprising a hydrophobic polymer and one or more skin active agents. Methods of making the composition and methods for treating skin are also disclosed.

### BACKGROUND

Skin is a unique and complex organ extending over the entire body. It is critical for protecting the body from external threats, controlling body temperature, and preventing loss of moisture. It also serves as a sensory organ and produces vitamin D. There are different types of skin at different locations of the body. For example, facial skin is different from that of the scalp, and even the skin on the palm of the hand is different than skin on the back of the hand.

Skin makes up the integumentary system, which includes three layers: the epidermis, the dermis, and the hypodermis. The epidermis is the outermost layer of skin and the first line of defense from external threats such as microbes. The human epidermis is principally composed of keratinocytes contains other types of cells including the melanocytes and the Langerhans' cells. Each of these cell types contribute, through their specific function, to the essential role played by the skin. Melanocytes provide color and tone characteristic of the skin. The epidermis is essentially waterproof and therefore helps maintain hydration and prevents the body from absorbing water when bathing. Unlike other cells, epidermal cells have a unique ability to regenerate and therefore can heal after being wounded.

The middle layer of skin that lies right below the epidermis, is the dermis, which includes connective tissue, collage, elastin, hair follicles lymph nodes, and sweat glands. The dermis provides a solid support for the epidermis. It is also its feeder layer. The dermis includes mainly of fibroblasts, but leukocytes, mast cells or tissue macrophages are also present. The dermis also contains blood vessels and nerve fibers. The acellular part (i.e., the area in between the cells) of the dermis is called extracellular matrix. The extracellular matrix of skin is composed of various extracellular components including proteins, in particular collagen fibers and elastin. Other extracellular matrix components of skin include glycosaminoglycans (e.g., hyaluronic acid, chondroitin sulfate, dermatan sulfate, keratan sulfate, heparan sulfate, etc.), proteoglycans (e.g., fibromodulin, decorin, biglycan, perlecan, heparan sulfate proteoglycan 2, agrin, versican, aggrecan, lumican, collagen type IX, collagen type XII, collagen type XIV, testican 1, testican 2, etc.) and various glycoproteins (e.g., fibrillin 1, thrombospondin-1 and -2, tenascin-C and -X, osteopontin, fibronectin, laminin-5 and -6, vitronectin, etc.). These extracellular components are synthesized by dermal fibroblasts, which make dermal fibroblasts the primary constituent in the structural assembly of the dermis.

The extracellular matrix is a highly heterogeneous amalgam of morphologically diverse architectural entities. It organizes and imparts structural integrity to individual tissues, in addition to modulating cell behavior by interacting with cell surface receptors and soluble growth factors. Dysfunctions and changes in components of the extracellular matrix can therefore interfere with both tissue integrity and cell performance. Dysfunctions and changes in components of the extracellular matrix of skin and mucosa in humans can lead to skin aging, skin atrophy, damaged skin, wounded skin, and other problems.

The hypodermis is the deepest layer of the skin and is mostly fat and connective tissue. It connects the skin to underlying fascia (tissue) of the bones and muscles. The hypodermis not always classified as an official layer of skin, but it nonetheless provides important functions like storing energy, insulating the body, and preventing the skin from sagging. The hypodermis houses fat-storing cells that function as energy reservoirs and act as a cushion against impact.

The skin ages over time and wrinkles, dark spots, and loss of elasticity and volume can ensue. Skin aging includes both chronological aging (endogenous aging), which is a natural aging that occurs over time due to natural biological processes, and degenerative aging due to outside influences such as exposure to damaging sunlight, pollution, lack of hydration, etc. Ultraviolet rays such as UVB having a wavelength in the range of 280 nm to 320 nm and are known to cause skin damage and accelerate aging. UVB rays stimulate reactive oxygen species (ROS) and free radicals in skin cells, which speed the intracellular signaling system inducing oxidative stress on biomolecules such as DNA, proteins, and lipids. Increasing the oxidative stress of skin cells may cause stimulation of keratinocytes of the epidermis or fibroblasts of the dermis, and through a series of intracellular signal transduction, the expression of genes such as matrix metalloproteinase (MMP), a collagen degrading enzyme, may be increased.

There are many treatments available for treating skin. For example, the importance of collagen in the aging process has led to the development of many collagen-containing topical products. Other components such as retinoic acid, vitamin C and hyaluronic acid are used in topical formulations to help stimulate collagen synthesis. Some cosmetic procedures, such as laser skin resurfacing, are used for reducing facial wrinkles and skin irregularities. The topical administration of proteins known to inhibit one or more signaling pathways that exhibit altered activity after UV exposure have been explored. However, there are difficulties associated with the use of whole proteins, which cannot be effectively delivered though the epidermis. Also, large proteins can be unstable, difficult to formulate, and not topically effective. Despite the number of product and treatments that have been proposed none of them satisfactory evoke the intrinsic repair and renewal mechanisms of the skin.

### SUMMARY OF THE DISCLOSURE

The instant disclosure is drawn to cosmetic compositions in the form of an oil-in-water dispersions. The compositions effectively incorporate and deliver skin active agents to the skin. This is achieved, at least in part, due to inclusion of a unique hydrophobic polymer, which is solubilized in an oil or an oil phase. The hydrophobic polymer helps stabilize the dispersion, reduces oil droplet size, and enhances penetration of skin active agents into the skin. Skin active agents can be difficult to formulate into cosmetic compositions and deliver into the skin. For example, many skin active agent suffer from solubility and stability problems. Even if skin active agents are successfully formulated for stability, a stable formulation does not necessarily ensure the skin active agents penetrates the skin. The oil-in-water emulsions of the instant case, however, are both stable and effective for delivering skin active agents deep into the skin.

Oil-in-water dispersions can become physically unstable due to causes such as creaming, sedimentation, flocculation, phase inversion, and coalescence. The tendency toward instability is dependent, at least in part, on droplet size, droplet distribution, amount and type of emulsifiers, and the mutual solubility between phases. Better emulsifier adsorption results in a reduction of interfacial tension, lowers the interface free energy, and thus favors emulsification and emulsion stability. The inventors found that the unique hydrophobic polymers of the instant emulsions positively influence (reduce) the interfacial tension and contribute to a robust and stable cosmetic composition that successfully incorporates and delivers skin active agents, in high amounts.

The cosmetic composition is an oil-in-water emulsion comprising:
(a) a hydrophobic polymer formed as a reaction product of a natural or food-derived oil and a methacrylate or acrylate polymer;
(b) one or more solvents capable of solubilizing the reaction product of (a);
(c) one or more skin active agents;
(d) one or more surfactants; and
(e) water.

The hydrophobic polymer is a unique reaction product of a natural or food-derived oil and an acrylate or methacrylate polymer. Preferably, the hydrophobic polymer is a reaction product of a methacrylate polymer and a natural or food-derived oil. The natural or food-derived oil is preferably a drying oil or a semi-drying oil. Nonlimiting examples include linseed oil, sunflower oil, tung oil, fish oil, cottonseed oil, soybean oil, or combinations thereof. The methacrylate polymer can be formed from methacrylate monomers, for example, monomers selected from isobutyl methacrylate, methyl methacrylate, ethyl methacrylate, n-butyl methacrylate, and combinations thereof. In a preferred embodiment, the hydrophobic polymer is formed from a natural or food-derived oil and an isobutyl methacrylate polymer and may preferably be an isobutyl methacrylate polymer.

In various embodiments, the hydrophobic polymer is the reaction product of about 50 to about 85 parts by weight of the natural or food-derived oil and about 15 to about 50 parts by weight of the methacrylate or acrylate polymer. More specifically, the hydrophobic polymer may be the reaction product of about 72 to about 77 parts by weight of the natural or food-derived oil and about 23 to about 28 parts by weight of a methacrylate polymer. For example, the hydrophobic polymer may be the reaction product of linseed oil and poly(isobutyl methacrylate) in a suitable solvent, such as, for example, 2,2,4-trimethyl-1, 3-pentanediol monoisobutyrate. Preferably, the reaction product is formed from about 72 to about 77% of linseed oil and about 23 to about 28% of isobutyl methacrylate polymer in a suitable solvent, such as 2,2,4-trimethyl-1, 3-pentanediol monoisobutyrate.

One or more solvents capable of solubilizing the hydrophobic polymer of (a) are used to solubilize the hydrophobic polymer. A single solvent may be used or a combination of solvents, wherein the combination of solvents is capable of solubilizing the hydrophobic polymer. In various embodiments, the one or more solvents capable of solubilizing the hydrophobic polymer have a dispersion component (D), a polar component (P), and a hydrogen bonding component (H), and a distance (Ra) per Hansen Solubility Parameters of less than or equal to 13.4 MPa^{0.5}, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
D₁ is 16.8 MPa^{0.5},
P₁ is 4.8 MPa^{0.5}, and
H₁ is 13.0 MPa^{0.5}.

Nonlimiting examples of solvents capable of solubilizing the hydrophobic polymer of (a) include polycitronellol acetate, caprylic/capric triglyceride, isododecane, isohexadecane, tetradecane, isopropyl myristate, isopropyl alcohol, octyldodecanol, ethanol, phenoxyethanol, castor oil, and mixtures thereof. Polycitronellol acetate, caprylic/capric triglyceride, isododecane, and mixtures thereof are particularly useful.

Surfactants include anionic surfactants, cationic surfactants, amphoteric (zwitterionic) surfactants, and nonionic surfactants. In various embodiments, the compositions of the instant disclosure include one or more anionic surfactants, and optionally, one or more nonionic surfactants. Furthermore, one or more of the surfactants may preferably be a biosurfactant.

Nonlimiting examples of biosurfactants include glycolipids (e.g., sophorolipids, rhamnolipids, cellobiose lipids, mannosylerythritol lipids and trehalose lipids), lipopeptides (e.g., surfactin, iturin, fengycin, arthrofactin and lichenysin), flavolipids, phospholipids (e.g., cardiolipins), fatty acid ester compounds, fatty acid ether compounds, and high molecular weight polymers such as lipoproteins, lipopolysaccharide-protein complexes, and polysaccharide-protein-fatty acid complexes. Preferably, at least one of the biosurfactants is a glycolipid. Nonlimiting examples of glycolipids include sophorolipids, rhamnolipids, trehalose lipids, mannosylerythritol lipids, and combinations thereof. Rhamnolipids are particularly preferred.

Nonlimiting examples of anionic surfactants include sulfate-based surfactants, glutamate surfactants, acyl taurates, alkanoyl isethionates, alkyl succinates, alkyl sulphosuccinates, N-alkoyl sarcosinates, alkyl phosphates, alkyl ether phosphates, alkyl ether carboxylates, alpha-olefin sulphonates, or combinations thereof. In various embodiments, the compositions of the instant disclosure include one or more acyl taurate surfactants.

A wide variety of skin active agents may be incorporated into the cosmetic compositions due in part to their surprising stability and versatility. For example, the oil phase of the cosmetic composition may include one or more lipophilic skin active agents. The aqueous phase may include one or hydrophilic skin active agents.

Nonlimiting examples of useful skin active agents include ceramides, ceramide precursors, cholesterol, cholesterol sulfate, b-glucan, carob seed extract, Eperua falcata extract, amino acids, niacinamide and its derivatives, hyaluronic acid and its derivatives allantoin, omega fatty acids, vitamins, vitamin precursors, retinoids including retinol, retinoic acid, tretinoin, isotretinoin, adapalene, or mixtures thereof.

In a preferred embodiment, the cosmetic composition includes one or more ceramides, for example, one or more ceramides selected from Ceramide EOP, Ceramide AS, Ceramide AP, Ceramide NS, Ceramide NP, Ceramide NH, Ceramide AH, Ceramide EOH, Ceramide EOS, Ceramide AdS, Ceramide NdS, and Ceramide EOdS, protein bound ceramides, phytosphingosine, sphingosine, ceramide precursors, or combinations thereof. In a preferred embodiment, the cosmetic composition includes Ceramide-EOP, Ceramide-NP, Cermamide-EP, more mixtures thereof.

The disclosure further relates to methods of incorporating skin active agent into cosmetic compositions, methods for stabilizing compositions comprising skin active agents, methods for delivering one or more skin active agent into the skin, and to methods for treating skin with the cosmetic compositions.

### BRIEF DESCRIPTION OF THE DRAWING

Implementation of the present technology is described, by way of example only, with reference to the attached FIGURE, wherein:

The FIGURE shows and image from Cryo-Scanning Electron Microscopy of a droplet containing skin active agents (ceramides) dispersed in an inventive oil-in-water dispersion.

It should be understood that the various aspects are not limited to the arrangements and instrumentality shown in the drawings.

### DETAILED DESCRIPTION OF THE DISCLOSURE

The cosmetic compositions are the form of oil-in-water emulsions that stably incorporate and deliver skin active agents, in high amounts, to the skin. Also described are methods for making and using the cosmetic compositions, including methods for treating skin. The cosmetic compositions are surprisingly versatile, robust, and effective.

The compositions typically include:
(a) a hydrophobic polymer that is as a reaction product of a natural or food-derived oil and a methacrylate or acrylate polymer;
(b) one or more solvents capable of solubilizing the hydrophobic polymer of (a);
(c) one or more skin active agents;
(d) one or more surfactants; and
(d) water;
   wherein the composition is an oil-in-water emulsion, preferably an oil-in-water dispersion.

The cosmetic composition may optionally include one or more additional components, for example, water soluble solvents, thickening agents, film forming polymers, miscellaneous ingredients (e.g., vitamins, antioxidants, etc.), cholesterol, fatty alcohols, fatty acids, botanical extracts, or mixtures thereof.

### (a) Hydrophobic Polymer

The hydrophobic polymer is a reaction product of a natural or food-derived oil (oil component) and an acrylate component. In particular, the natural or food-derived oil may be a drying oil, preferably linseed oil. The reaction product may include an isobutyl methacrylate backbone with a plurality of linseed oil side chains. Preferably, the reaction product is a product sold under the MYCELX® brand from MYCELX Technologies Corporation of Gainesville, Georgia. See U.S. Pat. No. 5,698,139 for a description of MYCELX materials, which is incorporated herein by reference in its entirety.

The hydrophobic polymer is comprised of an oil component and a polymer component, typically reacted in a solvent. In a preferred embodiment, the hydrophobic polymer is a reaction product of linseed oil and poly(isobutyl methacrylate), reacted in a solvent, such as 2,2,4-trimethyl-1, 3-pentanediol-monoisobutyrate as a solvent.

The oil component is derived from glycerin and carboxylic acids, such as linseed fatty acid to form monoglycerides, diglycerides, and triglycerides. The oil component is preferably derived from plant/vegetable or natural origin. Vegetable oils are obtained by cold pressing the seeds of a plant to obtain the oil contained therein. Of the vegetable oils, drying oils such as linseed and tung oil, semi-drying oils such as soybean and cotton seed oil, and non-drying oils such as coconut oil may be used as the oil component. The oil component typically forms about 72% to 77%, or most preferably, 74.62%, of the hydrophobic polymer (e.g., linseed oil/isobutyl methacrylate).

The polymer component may be derived from α and β-unsaturated carbonyl compounds. The polymer component is the resultant product of a monomer which is an ester of an acrylic acid, crotonic acid, isocrotonic acid, methacrylic acid, sorbic acid, cinnamic acid, maleic acid, fumaric acid, and methyl methacrylic acid. Nonlimiting examples of useful polymers which cover any number of reaction possibilities between the esters of such compounds include acrylate polymers, methyl methacrylate polymers, methyl/n-butyl methacrylate polymers, methacrylate copolymers, ethyl methacrylate, n-butyl methacrylate, isobutyl methacrylate, n-butyl/isobutyl methacrylate copolymers, or mixtures thereof.

The hydrophobic polymer is a reaction product typically formed in a liquid solvent able to dissolve or dilute the polymer component (poly(oil/polymer)) and the hydrophobic polymer. The solvent, or diluent should generally comprise any liquid or mixture of liquids that is able to dissolve or dilute hydrophobic polymer product. The solvent/diluent can control the evaporation, desired flow, and coalescing of the hydrophobic polymer. The solvent may be, for example, an aliphatic hydrocarbon, aromatic hydrocarbon, alcohols, ketones, ethers, aldehydes, phenols, carboxylic acids, carboxylates, synthetic chemicals and naturally occurring substances. Preferably the solvent is 2,2,4-trimethyl-1,3-pentanediol-monoisobutyrate. Hydrophobic polymers according to the instant disclosure and methods for making them are described, for example, in U.S. Pat. Nos. 5,437,793, 5,698,139, 5,837,146, 5,961,823, 6,180,010, 6,475,393, and 6,805,727, which are incorporated herein by reference in their entireties. The preferred hydrophobic polymer may be designated as poly(linseed oil/isobutyl methacrylate).

The amount of hydrophobic polymer in the cosmetic composition will vary but may be from about 0.01 to about 15 wt. %, based on the total weight of the cosmetic composition. In further embodiments, the total amount of the hydrophobic polymer in the cosmetic composition is from about 0.01 to about 10 wt. %, 0.01 about 0.1 to about 8 wt. %, about 0.01 to about 5 wt. %, about 0.01 to about 3 wt. %, about 0.1 to about 15 wt. %, about 0.1 to about 10 wt. %, about 0.1 about 0.1 to about 8 wt. %, about 0.1 to about 5 wt. %, about 0.1 to about 3 wt. %, about 0.5 to about 15 wt. %, about 0.5 to about 12 wt. %, about 0.5 to about 10 wt. %, about 0.5 to about 8 wt. %, about 0.5 to about 5 wt. %, or about 0.5 to about 3 wt. %, based on the total weight of the cosmetic composition.

### (b) Solvent Capable of Solubilizing (a)

The oil phase of the emulsion includes the hydrophobic polymer of (a) dissolved in one or more solvents capable of solubilizing the hydrophobic polymer. The one or more solvent may include one or more solvents used to generate or solubilize the hydrophobic polymer, e.g., 2,2,4-trimethyl-1,3-pentanediol-monoisobutyrate. The one or more solvents may be a single solvent or a plurality of solvents. For example, in various embodiments, solvents capable of solubilizing the hydrophobic polymer of (a) have a dispersion component (D), a polar component (P), a hydrogen bonding component (H), and a distance (Ra) of less than or equal to 13.4 MPa^{0.5} per the Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
D₁ is 16.8 MPa^{0.5},
P₁ is 4.8 MPa^{0.5}, and
H₁ is 13.0 MPa^{0.5}.

In a preferred embodiment, the one or more solvents have a dispersion component (D), a polar component (P), a hydrogen bonding component (H), and a distance (Ra) of less than or equal to 9.9 MPa^{0.5} per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
D₁ is 16.4 MPa^{0.5},
P₁ is 5.0 MPa^{0.5}, and
H₁ is 11.7 MPa^{0.5}.

The solvent may be an oil. The term “oil” is intended to mean a non-aqueous compound, non-miscible in water, and liquid at 25° C. and atmospheric pressure (760 mmHg; 1.013×10⁵ Pa). The solvent may be a non-silicone oil (e.g., an oil that does not contain silicon atoms, and in particular does not contain Si—O groups). Nonlimiting examples of the one or more solvents include caprylic/capric triglyceride, isopropyl myristate, polycitronellol acetate, isododecane, and mixtures thereof. The solvents may include acetone. The solvents may include oleic acid. The solvents may include an oleic acid containing oil (such as a vegetable oil). Table 1, below, shows values of D, P, and H, as well as Ra for allowable ranges as well as preferred ranges, for several solvents.

TABLE 1

| | | | | Ra | Ra |
|---|---|---|---|---|---|
| Solvent (b) | D | P | H | (Allowable Range) | (Preferred Range) |
| Ethanol | 15.8 | 8.8 | 19.4 | 7.81 | 8.67 |
| Octyldodecanol | 16.1 | 2.2 | 7.4 | 6.33 | 5.17 |
| Isopropyl | 15.9 | 2.1 | 2.8 | 10.70 | 9.41 |
| Myristate | | | | | |
| Isopropyl Alcohol | 15.8 | 6.1 | 6.4 | 7.02 | 5.54 |
| Phenoxyethanol | 17.8 | 5.7 | 14.3 | 2.55 | 3.88 |
| CCTG | 18.22 | 5.39 | 14.74 | 3.38 | 4.76 |
| Castor Oil | 15.9 | 4.6 | 12 | 2.07 | 1.12 |
| Polycitronellol | 16.4 | 3 | 4.2 | 9.02 | 7.76 |
| Acetate | | | | | |
| Acetone | 15.5 | 10.4 | 7 | 8.61 | 7.38 |
| Oleic Acid | 16 | 2.8 | 6.2 | 7.27 | 5.98 |

In some embodiments, if oleic acid is utilized, at least some of the oleic acid may be provided by a vegetable oil. The vegetable oil may be a seed or nut oil. The vegetable oil may have an oleic acid content of at least 20% by weight of the vegetable oil. The vegetable oil may include sunflower oil, soybean oil, macadamia nut oil, and/or avocado oil. In some embodiments, the composition may include macadamia nut oil, and may be free, or substantially free, of other vegetable oils.

For purposes of the instant disclosure, the one or more of the solvents capable of solubilizing the hydrophobic polymer of (a) may not individually solubilize the hydrophobic polymer but when combined with other solvents, the combination solubilizes the hydrophobic polymer. Thus, when referring to a total amount of one or more solvents capable of solubilizing the hydrophobic polymer, the inclusion of all solvents that in combination solubilize the hydrophobic polymer is intended, even if one or more solvents in the combination do not individually solubilize the hydrophobic polymer.

Nonlimiting examples of solvents useful for solubilizing the hydrophobic polymer of (a), individually or in combination with other solvents, include polycitronellol acetate, caprylic/capric triglyceride, isododecane, isohexadecane, tetradecane, isopropyl myristate, octyldodecanol, ethanol, phenoxyethanol, castor oil, and mixtures thereof. In a preferred embodiment, at least one of the one or more solvents capable of solubilizing the hydrophobic polymer of (a) are selected from caprylic/capric triglyceride, polycitronellol acetate, isododecane, and mixtures thereof. In another preferred embodiment, at least one of the one or more solvents capable of solubilizing the hydrophobic polymer of (a) is polycitronellol acetate.

Nonlimiting solvent that individually or in combination with other solvents are useful for solubilizing the hydrophobic polymer of (a) include dioctylcyclohexane, mineral oil, isocetyl palmitate, isocetyl palmitate, cyclopentasiloxane, dicaprylyl carbonate, octyl isostearate, trimethylhexyl isononanoate, 2-ethylhexyl isononanoate, dicaprylyl ether, dihexyl carbonate, polydecene, octyl cocoate, isodecyl neopentanoate, isohexy decanoate, isodecyl octanoate, dihexyl ether, isododecane, isodecyl 3,5,5 trimethyl hexanoate, oleyl erucate, Passiflora incarnata oil, jojoba oil, octyl palmitate, macadamia nut oil, isopropyl stearate, rapeseed oil, hexyl decanol, isotridecyl 3,5,5 trimethylhexanonanoate, polycitronellol acetate, mixed decanoyl and octanoyl glycerides, 2-ethylhexanoic acid, 3,5,5 trimethyl ester, cetystearyl octanoate, dimethicone, isopropyl palmitate, octyldodecanol, dioctyl adipate, isopropyl myristate, octyl palmitate (2-ethylhexyl palmitate), octyldodeceyl myristate, butyl octanoic acid, isopropyl stearate, caprylic/capric triglycerides, isopropyl isostearate, Jojoba oil, cyclomethicone, groundnut oil, almond oil, sunflower oil, decyl oleate, avocado oil, olive oil, dibutyl adipate, castor oil, calendula oil, wheatgerm oil, decyl oleate, avocado oil, calendula oil, propylene glycol monoisostearate, cocoglycerides, butylene glycol caprylate/caprate, C12-15 alkyl benzoate, caprylic/capric diglyceryl succinate, caprylic/capric triglyceride, cetearyl isonoanoate, cetearyl octanoate, cetyl dimethicone, coco-caprylate/caprate, cocoglycerides, Di-C12-13 alkyl tartrate, dibutyl adipate, dicaprylyl carbonate, dicaprylyl ether, hexyl decanol, hydrogenated polyisobutene, isoeicosane, isohexadecane, isopropyl palmitate, isopropyl stearate, octyl cocoate, octyl isostearate, octyl octanoate, octyl palmitate, octyl stearate, octyl dodecanol, octyldodecyl myristate, isopropyl stearate, pentaerythrityl tetraisostearate, phenyl trimethicone, polydecene, propylene glycol dicaprylate/dicaprate, stearyl heptanoate, tricaprylin, tridecyl stearate, tridecyl trimellitate, triisostearin, or combinations thereof.

Additionally useful solvents include hydrocarbon-based oils of plant origin, such as liquid triglycerides of fatty acids containing from 4 to 10 carbon atoms, for instance heptanoic or octanoic acid triglycerides, or alternatively, for example, sunflower oil, corn oil, soybean oil, marrow oil, grapeseed oil, sesameseed oil, hazelnut oil, apricot oil, macadamia oil, arara oil, coriander oil, castor oil, avocado oil, caprylic/capric acid triglycerides, for instance those sold by the company Stearineries Dubois or those sold under the names MIGLYOL 810, 812 and 818 by the company Dynamit Nobel, jojoba oil, shea butter oil and caprylyl glycol; synthetic esters and ethers, especially of fatty acids, for instance Purcellin oil, 2-octyldodecyl stearate, 2-octyldodecyl erucate, isostearyl isostearate; hydroxylated esters, for instance isostearyl lactate, octyl hydroxystearate, octyldodecyl hydroxystearate, diisostearyl malate or triisocetyl citrate; fatty alcohol heptanoates, octanoates or decanoates; polyol esters, for instance propylene glycol dioctanoate, neopentyl glycol diheptanoate and diethylene glycol diisononanoate; and pentaerythritol esters, for instance pentaerythrityl tetraisostearate, or isopropyl lauroyl sarcosinate, sold especially under the trade name ELDEW SL 205 by the company Ajinomoto; linear or branched hydrocarbons, of mineral or synthetic origin, such as volatile or non-volatile liquid paraffins, and derivatives thereof, petroleum jelly, polydecenes, isohexadecane, isododecane, hydrogenated polyisobutene such as Parleam oil, or the mixture of n-undecane (C₁₁) and of n-tridecane (C₁₃) sold under the reference CETIOL UT by the company Cognis; fluoro oils that are partially hydrocarbon-based and/or silicone-based, for instance those described in document JP-A-2 295 912; silicone oils, for instance volatile or non-volatile polymethylsiloxanes (PDMS) with a linear or cyclic silicone chain, which are liquid or pasty at room temperature, in particular volatile silicone oils, especially cyclopolydimethylsiloxanes (cyclomethicones) such as cyclohexadimethylsiloxane and cyclopentadimethylsiloxane; polydimethylsiloxanes comprising alkyl, alkoxy or phenyl groups, which are pendent or at the end of a silicone chain, these groups containing from 2 to 24 carbon atoms; phenyl silicones, for instance phenyl trimethicones, phenyl dimethicones, phenyltrimethylsiloxydiphenylsiloxanes, diphenyl dimethicones, diphenylmethyldiphenyltrisiloxanes or 2-phenylethyl trimethylsiloxy silicates, and polymethylphenylsiloxanes; mixtures thereof.

Additional specific examples solvents include cocoglyceride, cyclomethicone, dimethicone, dicapryl maleate, caprylic/capric triglyceride, isopropyl myristate, octyl stearate, isostearyl linoleate, lanolin oil, coconut oil, cocoa butter, olive oil, avocado oil, aloe extracts, jojoba oil, castor oil, fatty acids such as oleic acid, stearic acid, fatty alcohols such as cetyl alcohol, hexadecyl alcohol, diisopropyl adipate, hydroxybenzoate esters, benzoic acid esters of C₉-C₁₅ alcohols, isononyl iso-nonanoate, alkanes, mineral oil, silicone, dimethyl polysiloxane, ether, polyoxypropylene butyl ether, polyoxypropylene cetyl ether, C₁₂-C₁₅ alkyl benzoate, aryl alkyl benzoate, isopropyl lauroyl sarcosinate, or mixtures thereof.

The total amount of the one or more solvents capable of solubilizing the hydrophobic polymer of (a) in the cosmetic composition will vary. In some instances, the oil phase of the oil-in-water emulsions is predominately the one or more solvents capable of solubilizing the hydrophobic polymer. For example, the one or more solvents capable of solubilizing the hydrophobic polymer of (a) may be in amounts from about 10 to about 70 wt. %, based on the total amount of the cosmetic composition. In further embodiments, the total amount of the one or more solvents capable of solubilizing the hydrophobic polymer may be in amounts from about 10 to about 60 wt. %, about 10 to about 50 wt. %, about 10 to about 40 wt. %, about 10 to about 30 wt. %, about 20 to about 70 wt. %, about 20 to about 60 wt. %, about 20 to about 50 wt. %, about 20 to about 40 wt. %, about 30 to about 70 wt. %, about 30 to about 60 wt. %, about 30 to about 50 wt. %, about 30 to about 40 wt. %, about 40 to about 70 wt. %, about 40 to about 60 wt. %, or about 40 to about 60 wt. %, based on the total weight of the cosmetic composition.

The oil-in-water emulsions forming the cosmetic composition do not require a high amount of an oil phase, which is dispersed in droplets throughout the aqueous phase. Thus, in various embodiments, the cosmetic composition can include from about 0.05 to about 20 wt. % of the one or more solvents capable of solubilizing the hydrophobic polymer, based on the total weight of the cosmetic composition. In further embodiments, the total amount of the one or more solvents capable of solubilizing the hydrophobic polymer can be from about 0.05 to about 15 wt. %, about 0.05 to about 10 wt. %, about 0.05 to about 8 wt. %, about 0.05 to about 5 wt. %, about 0.05 to about 3 wt. %, about 0.1 to about 20 wt. %, about 0.1 to about 15 wt. %, about 0.1 to about 10 wt. %, about 0.1 to about 8 wt. %, about 0.1 to about 5 wt. %, about 0.1 to about 3 wt. %, about 0.5 to about 20 wt. %, about 0.5 to about 15 wt. %, about 0.5 to about 10 wt. %, about 0.5 to about 8 wt. %, about 0.5 to about 5 wt. %, or about 0.5 to about 3 wt. %, about 2 to about 20 wt. %, about 2 to about 15 wt. %, about 2 to about 10 wt. %, about 5 to about 20 wt. %, about 5 to about 10 wt. %, about 10 to about 30 wt. %, or about 15 to about 30 wt. % based on the total weight of the cosmetic composition.

### (c) Skin Active Agents

Skin active agents are ingredients included in the cosmetic composition to benefit, treat, or improve the health, appearance, or integrity of the skin. Skin active ingredients are often classified according to their function. Nonlimiting examples include anti acne compounds, anti-inflammatory compounds, skin renewal compounds, compounds that improve skin barrier function, depigmenting compounds, anti-wrinkle compounds, anti-aging compounds, and the like. For example, skin active agents include rosacea inhibitory agents (e.g., metronidazole, sulfacetamide, sodium sulfacetamide, sulfur, dapson, doxycycline, minocycline, clindamycin, clindamycin phosphate, erythromycin, tetracylclines, azelaic acid, calcium dobesilate, maleic acid, and any compatible combinations thereof); α-adrenergic receptor agonists (e.g., clonidine, amphetamine, doxtroamphetamine, apraclonidine, dipivefrin, α-methyldopa, oxymetazoline, oxymetazoline hydrochloride, methoxamine, metaraminol, medetomidine, dexmedetomidine, ethylnorepinephrine, guanfacine, guanabenz, phenylephrine, phenylephrine hydrochloride, ephedrine, epinine, epinephrine, ethylnorepinephrine, levarterenol, lofexidine, norepinephrine, norphenylephrine, norephedrine, phenylpropanolamine, pemoline, propylhexadrine, pseudoephedrine, methamphetamine, α-methylnorepinephrine, methylphenidate, mephentermine, midodrine, mivazerol, moxonidine, desglymidodrine, tetrahydrozoline, tetrahydrozoline hydrochloride, cirazoline, amidephrine, brimonidine, brimonidine tartrate, naphazoline, isoproterenol, xylazine, xylometazoline, and/or tizanidine); chemicals and botanical extracts with vasoconstrictor properties including, but not limited to, corticosteroids, ephedrine, pseudoephedrine, caffeine, and/or escin; ephedra, phedra sinica, hamamelis viginiana, Hydrastis canadensis, Lycopus virginicus, aspidosperma quebracho, cytisus scoparius, raphanus sativus linn (radish leave extracts), horse chestnut extracts, etc.

In a preferred embodiment, the skin active agents are selected from ceramides, ceramide precursors, free fatty acids, linoleic/oleic acids, cholesterol, cholesterol sulfate, b-glucan, carob seed extract, Eperua falcata extract, amino acids, niacinamide and its derivatives, hyaluronic acid & its derivatives, glycerin, allantoin, squalane, omega fatty acids, or combinations thereof.

Additional nonlimiting examples include chemicals or botanical extracts with anti-inflammatory properties (e.g., corticosteroids (for short term use)), non-steroidal anti-inflammatory drugs, linoleic acid, linolenic acid, bisabolol, glycyrrhetinic acid, glycerin, plant extracts with anti-inflammatory properties (i.e., tea extracts, chamomile extracts), anti-inflammatory interleukins (e.g., II-1ra); isoprenylcystein analogues (i.e., N-acetyl-S-farnesyl-L-cysteine), aromatic aldehydes with anti-inflammatory properties (e.g., 4-ethoxy benzaldehyde), etc., as well as any compatible combinations thereof); chemicals or botanical extracts; chemicals or botanical extracts with anti-microbial properties (e.g., antibiotics including, but not limited to gentamicin, penicillins, cephalosporins, quinolones, ciprofloxacin, and/or novobiocin); chemicals or botanical extracts with anti-fungal properties (e.g., ketoconazole, naftifine hydrochloride, oxiconazole nitrate, sulconazole nitrate, urea, terbinafine hydrochloride, selenium sulfide, etc.); chemicals or botanical extracts with anti-dandruff properties; chemicals or botanical extracts with anti-seborrheic properties; keratolytic agents or botanical extracts with keratolytic properties (i.e., alpha-hydroxy acids; beta-hydroxy acids, poly-hydroxy acids, urea, salicylic acid, etc.); chemicals with astringent properties; serine protease inhibitors; saturated dicarboxylic acids; alpha hydroxy acids (e.g., glycolic acids, lactic acid, malic acid, citric acid, tartaric acid, etc.); beta hydroxy acids (e.g., carnitine, 3-hydroxybutyric acid, 3-hydroxypropionic acid, β-hydroxy β-methylbutyric acid, salicylic acid, etc.).

Additional nonlimiting examples of skin active agents include retinoic acid, tretinoin, isotretinoin, adapalene, retinol, and/or derivatives; benzoyl peroxide; dapsone; kinetin (N6-furfuryladenine) and derivatives (e.g., furfurylaminotetrahydro-pyranyladenine); niacinamide (nicotinamide), and combinations thereof. Even further nonlimiting examples of skin active agents include metronidazole, sulfacetamide, sodium sulfacetamide, sulfur, tetracylines, doxycycline, clindamycin, clindamycin phosphate, erythromycin, and/or minocycline, azelaic acid, calcium dobesilate, caffeine, theobromine, theophylline and/or a derivative thereof (i.e., xanthines), vitamin A, vitamin B1, vitamin B2, vitamin B3, vitamin B5, vitamin B6, vitamin B7, vitamin B9, vitamin B12, vitamin C, vitamin D, vitamin E and vitamin K, creatine, carnitine, and essential fatty acids such as linoleic acid and/or linolenic acid, zinc salts such as, for example, zinc sulfate, zinc chloride, zinc glycinate, zinc gluconate, zinc-histidine, zinc L-2-pyrrolidone-5-carboxylate (zinc PCA), zinc salt of linoleic acid, zinc salt of linolenic acid, zinc salt of azelaic acid, zinc peptides, zinc oxide, copper salts including, but not limited to, copper sulfate, copper chloride, copper glycinate, copper gluconate, copper-histidine, copper L-2-pyrrolidone-5-carboxylate (copper PCA), copper salt of linoleic acid, copper salt of linolenic acid, copper salt of azelaic acid, copper peptides,

In various embodiments, the one or more skin active agents are optionally selected from madecassoside, retinoic acid, benzoyl peroxide, sulfur, vitamin B6 (pyridoxine or) chloride, selenium, samphire, cinnamon extract blends, zinc gluconate, zinc pyrrolidonecarboxylate (or zinc pidolate), zinc lactate, zinc aspartate, zinc carboxylate, zinc salicylate, zinc cysteate, copper and copper pidolate as Cuivridone Solabia, extracts from plants of Arnica montana, Cinchona succirubra, Eugenia caryophyllata, Humulus lupulus, Hypericum perforatum, Mentha pipenta, Rosmarinus officinalis, Salvia officinalis and Thymus vulgaris, extracts of meadowsweet (Spiraea ulmaria), and mixtures thereof.

In various embodiments, one or more skin active agents may be selected from adenosine, 2-[4-(2-hydroxyethyl)piperazin-1-yl]ethanesulfonic acid (HEPES), hyaluronic acid, lanolin, citric acid, malic acid, lactic acid, tartaric acid, salicylic acid, vitamin C, a vitamin, a retinoid, retinal, retinoic acid, a carotenoid, an amino acid, a protein, an enzyme, and a coenzyme. In some cases the active ingredient is adenosine.

The skin active agent may be selected from lactic acid, hyaluronic acid, AHA, BHA, sodium pidolate, serine, sodium lactate, ectoin and its derivatives, chitosan and its derivatives, collagen, plankton, an extract of Imperata cylindra sold under the name Moist 24 by Sederma, homopolymers of acrylic acid as Lipidure-HM of NOF Corporation, beta-glucan and in particular sodium carboxymethyl beta-glucan Mibelle-AG-Biochemistry, a mixture of oils passionflower, apricot, corn, and rice bran sold by Nestle under the name NUTRALIPIDS, a C-glycoside derivatives, in particular the C-13-D-xylopyranoside-2-hydroxypropane, for example, as the product produced by the company Chimex under the trade name “MEXORYL SBB”, a rose hip oil marketed by Nestle, a micro-algae extract Prophyridium cruentum enriched with zinc, marketed under the name by VINCIENCE ALGUALANE ZINC spheres of collagen and chondroitin sulfate of marine origin (Atelocollagen) sold by the company Engelhard Lyon under the name MARINE FILLING SPHERES, hyaluronic acid spheres such as those marketed by Engelhard Lyon, and arginine.

Depigmenting agents include vitamin C and its derivatives and especially vitamin CG, CP and 3-O ethyl vitamin C, alpha and beta arbutin, ferulic acid, lucinol and its derivatives, kojic acid, resorcinol and derivatives thereof, tranexamic acid and derivatives thereof, gentisic acid, homogentisic, methyl gentisate or homogentisate, dioic acid, D pantheteine calcium sulphonate, lipoic acid, ellagic acid, vitamin B3, linoleic acid and its derivatives, ceramides and their counterparts, derived from plants such as chamomile, bearberry, the aloe family (vera, ferox, bardensis), mulberry, skullcap, a water kiwi fruit (Actinidia chinensis) marketed by Gattefosse, an extract of Paeonia suffruticosa root, such as that sold by Ichimaru Pharcos under the name Liquid Botanpi Be an extract of brown sugar (Saccharum officinarum) such as molasses extract marketed by Taiyo Kagaku under the name Liquid Molasses, without this list being exhaustive. Particular depigmenting agents include vitamin C and its derivatives and especially vitamin CG, CP and 3-O ethyl vitamin C, alpha and beta arbutin, ferulic acid, kojic acid, resorcinol and derivatives, D pantheteine calcium sulfonate, lipoic acid, ellagic acid, vitamin B3, a water kiwi fruit (Actinidia chinensis) marketed by Gattefosse, an extract of Paeonia suffruticosa root, such as that sold by the company Ichimaru Pharcos under the name Botanpi Liquid B.

Useful skin active agents include adenosine and its derivatives and retinol and its derivatives such as retinol palmitate, ascorbic acid and its derivatives such as magnesium ascorbyl phosphate and ascorbyl glucoside; tocopherol and derivatives thereof such as tocopheryl acetate, nicotinic acid and its precursors such as nicotinamide; ubiquinone; glutathione and precursors thereof such as L-2-oxothiazolidine-4-carboxylic acid, the compounds C-glycosides and their derivatives as described in particular in EP-1345919, in particular C-beta-D-xylopyranoside-2-hydroxypropane as described in particular in EP-1345919, plant extracts including sea fennel and extracts of olive leaves, as well as plant and hydrolysates thereof such as rice protein hydrolysates or soybean proteins; algal extracts and in particular laminaria, bacterial extracts, the sapogenins such as diosgenin and extracts of Dioscorea plants, in particular wild yam, comprising: the a-hydroxy acids, f3-hydroxy acids, such as salicylic acid and n-octanoyl-5-salicylic oligopeptides and pseudodipeptides and acyl derivatives thereof, in particular acid {2-[acetyl-(3-trifluoromethyl-phenyl)-amino]-3-methyl-}acetic acid and lipopeptides marketed by the company under the trade names SEDERMA Matrixyl 500 and Matrixyl 3000; lycopene, manganese salts and magnesium salts, especially gluconates, and mixtures thereof.

As adenosine derivatives include especially non-phosphate derivatives of adenosine, such as in particular the 2′-deoxyadenosine, 2′,3′-adenosine isopropoylidene; the toyocamycine, 1-methyladenosine, N-6-methyladenosine; adenosine N-oxide, 6-methylmercaptopurine riboside, and the 6-chloropurine riboside.

Other derivatives include adenosine receptor agonists such as adenosine adenosine phenylisopropyl (“PIA”), 1-methylisoguanosine, N6-cyclohexyladenosine (CHA), N6-cyclopentyladenosine (CPA), 2-chloro-N6-cyclopentyladenosine, 2-chloroadenosine, N6-phenyladenosine, 2-phenylaminoadenosine, MECA, N 6-phenethyladenosine, 2-p-(2-carboxy-ethyl) phenethyl-amino-5′- -N-ethylcarboxamido adenosine (CGS-21680), N-ethylcarboxamido-adenosine (NECA), the 5′(N-cyclopropyl)-carboxamidoadenosine, DPMA (PD 129.944) and metrifudil.

In one embodiment the composition comprises an active ingredient that addresses oily skin. These actives can be sebo-regulating or antiseborrhoeic agents capable of regulating the activity of sebaceous glands. These include: retinoic acid, benzoyl peroxide, sulfur, vitamin B6 (pyridoxine or) chloride, selenium, samphire—the cinnamon extract blends, tea and octanoylglycine such as—15 Sepicontrol A5 TEA from Seppic—the mixture of cinnamon, sarcosine and octanoylglycine marketed especially by Seppic under the trade name Sepicontrol A5—zinc salts such as zinc gluconate, zinc pyrrolidonecarboxylate (or zinc pidolate), zinc lactate, zinc aspartate, zinc carboxylate, zinc salicylate 20, zinc cysteate; —derivatives particularly copper and copper pidolate as Cuivridone Solabia—extracts from plants of Arnica montana, Cinchona succirubra, Eugenia caryophyllata, Humulus lupulus, Hypericum perforatum, Mentha pipenta 25 Rosmarinus officinalis, Salvia officinalis and Thymus vulgaris, all marketed for example by Maruzen—extracts of meadowsweet (Spiraea ulmaria), such as that sold under the name Sebonormine by Silab—extracts of the alga Laminaria saccharina, such as that sold under the 30 name Phlorogine by Biotechmarine—the root extracts of burnet mixtures (Sanguisorba officinalis/Poterium officinale), rhizomes of ginger (Zingiber officinalis) and cinnamon bark (Cinnamomum cassia), such as that sold under the name SEBUSTOP by Solabia—extracts of flaxseed such as that sold under the name LINUMINE by Lucas Meyer—Phellodendron extracts such as those sold under the name Phellodendron extract BG by Maruzen or Oubaku liquid B by Ichimaru Pharcos—of argan oil mixtures extract of Serenoa serrulata (saw palmetto) extract and sesame seeds such as that sold under the name REGU SEB by Pentapharm—mixtures of extracts of willowherb, of Terminalia chebula, nasturtium and of bioavailable zinc (microalgae), such as that sold under the name SEBORILYS GREEN TECH; —extracts of Pygeum afrianum such as that sold under the name PYGEUM AFRIANUM STEROLIC LIPID EXTRACT by Euromed—extracts of Serenoa serrulata such as those sold under the name VIAPURE SABAL by Actives International, and those sold by the company Euromed—of extracts of plantain blends, Berberis aquifolium and sodium salicylate 20 such as that sold under the name SEBOCLEAR RAHN—extract of clove as that sold under the name CLOVE EXTRACT POWDER by Maruzen—argan oil such as that sold under the name LIPOFRUCTYL LABORATORIES SEROBIOLOGIQUES; 25—lactic protein filtrates, such as that sold under the name NORMASEB by Sederma—the seaweed laminaria extracts, such as that sold under the name LAMINARGHANE by Biotechmarine—oligosaccharides seaweed Laminaria digitata, such as that sold under the name PHYCOSACCHARIDE 30 AC by the company Codif—extracts of sugar cane such as that sold under the name POLICOSANOL by the company Sabinsa, the sulfonated shale oil, such as that sold under the name ICHTYOL PALE by Ichthyol—extracts of ‘meadowsweet (Spiraea ulmaria) such as that sold under the name CYTOBIOL ULMAIRE by societeLibiol—sebacic acid, especially sold in the form of a sodium polyacrylate gel under the name SEBOSOFT by Sederma—glucomannans extracted from konjac tuber and modified with alkylsulfonate chains such as that sold under the name BIOPOL BETA by Arch Chemical—extracts of Sophora angustifolia, such as those sold under the name SOPHORA POWDER or Sophora extract by Bioland—extracts of cinchona bark succirubra such as that sold under the name RED BARK HS by Alban Muller—extracts of Quillaja saponaria such as that sold under the name 15 PANAMA WOOD HS by Alban Muller—glycine grafted onto an undecylenic chain, such as that sold under the name LIPACIDE UG OR by SEPPIC—the mixture of oleanolic acid and nordihydroguaiaretic acid, such as that sold under the form of a gel under the name AC.NET by Sederma; 20—phthalimidoperoxyhexanoic acid—citrate tri (C12-C13) sold under the name COSMACOL ECI by Sasol; trialkyl citrate (C14-C15) sold under the name COSMACOL ECL by Sasol—10-hydroxydecanoic acid, including mixtures acid-hydroxydecanoic October 25, sebacic acid and 1,10-decandiol such as that sold under the name ACNACIDOL BG by Vincience and mixtures thereof.

Other skin active agents useful herein include those selected from N-acetyl D-glucosamine, panthenol (e.g., DL panthenol available from Alps Pharmaceutical Inc.), tocopheryl nicotinate, benzoyl peroxide, 3-hydroxy benzoic acid, flavonoids (e.g., flavanone, chalcone), farnesol, phytantriol, glycolic acid, lactic acid, 4-hydroxy benzoic acid, acetyl salicylic acid, 2-hydroxybutanoic acid, 2-hydroxypentanoic acid, 2-hydroxyhexanoic acid, cis-retinoic acid, trans-retinoic acid, retinol, retinyl esters (e.g., retinyl propionate), phytic acid, N-acetyl-L-cysteine, lipoic acid, tocopherol and its esters (e.g., tocopheryl acetate: DL-α-tocopheryl acetate available from Eisai), azelaic acid, arachidonic acid, tetracycline, ibuprofen, naproxen, ketoprofen, hydrocortisone, acetominophen, resorcinol, phenoxyethanol, phenoxypropanol, phenoxyisopropanol, 2,4,4′-trichloro-2′-hydroxy diphenyl ether, 3,4,4′-trichlorocarbanilide, octopirox, lidocaine hydrochloride, clotrimazole, miconazole, ketoconazole, neomycin sulfate, theophylline, and mixtures thereof.

In a preferred embodiment, the cosmetic composition includes one or more skin active agents selected from free ceramides, e.g., Ceramide EOP, Ceramide AS, Ceramide AP, Ceramide NS, Ceramide NP, Ceramide NH, Ceramide AH, Ceramide EOH, Ceramide EOS, Ceramide AdS, Ceramide NdS, and Ceramide EOdS, protein bound ceramides, Phytosphingosine, Sphingosine, ceramide precursors, fatty acids, fatty alcohols, cholesterol, cholesterol sulfate, or combinations thereof.

In various embodiments, the total amount of skin active agents in the cosmetic composition of the disclosure, may be in an amount greater than zero to about 9 wt. %, greater than zero to about 8 wt. %, greater than zero to about 7 wt. %, greater than zero to about 6 wt. %, greater than zero to about 5 wt. %, greater than zero to about 4 wt. %, greater than zero to about 3 wt. %, greater than zero to about 2 wt. %; about 10 ppm to about 10 wt. % (100,000 ppm), about 10 ppm to about 5 wt. % (50,000 ppm), about 10 ppm to about 2.5 wt. % (25,000 ppm), about 10 ppm to about 1 wt. % (10,000 ppm), about 10 ppm to about 0.5 wt. % (5,000 ppm), about 10 ppm to about 0.3 wt. % (3,000 ppm), about 10 ppm to about 0.2 wt. % (2,000 ppm), about 10 ppm to about 0.1 wt. % (1,000 ppm), about 10 ppm to 500 ppm; about 0.1 to about 10 wt. %, about 0.1 to about 5 wt. %, about 0.1 to about 2.5 wt. %, about 0.1 to about 1 wt. %, about 0.1 to about 0.5 wt. %; about 1 to about 10 wt. %, about 1 to about 8 wt. %, about 1 to about 6 wt. %, about 1 to about 5 wt. %, about 1 to about 4 wt. %, about 1 to about 3 wt. %; about 2 to about 10 wt. %, about 2 to about 8 wt. %, about 2 to about 6 wt. %, about 2 to about 5 wt. %, about 2 to about 4 wt. %; about 3 to about 10 wt. %, about 3 to about 8 wt. %, about 3 to about 6 wt. %, about 3 to about 5 wt. %; about 4 to about 10 wt. %, about 4 to about 8 wt. %, or about 4 to about 6 wt. %, based on the total weight of the cosmetic composition.

### (d) Surfactant

For purposes of the instant disclosure, the term “surfactant” includes emulsifiers and detergents. Surfactants, or surface-active agents, are compounds that lower the surface tension between two liquids or between a liquid and a solid. Surfactants are amphiphilic, meaning that they contain hydrophilic (water-loving) head groups and hydrophobic (water-hating, or oil-loving) tails. Surfactants adsorb at the interface between oil and water, thereby decreasing the surface tension.

For purposes of the instant disclosure, an “emulsifier” is a surfactant that stabilizes emulsions. Emulsifiers coat droplets within an emulsion and prevent them from coming together, or coalescing. An “emulsion” is a mixture of two or more liquids, with or without an emulsifier, that are normally immiscible. One of the liquids, the “dispersed phase,” forms droplets in the other liquid, the “continuous phase.”

A “detergent” is a surfactant that has cleaning properties in dilute solutions and is typically anionic.

The surfactants can be anionic, cationic, amphoteric (zwitterionic), or nonionic. Preferably, the emulsions of the instant case include one or more surfactants selected from anionic surfactants, amphoteric (zwitterionic) surfactants, nonionic surfactants, or mixtures thereof. In various embodiments, the emulsions are preferably free or essentially free from cationic surfactants. In other embodiments, the emulsions include one or more cationic surfactants. In preferred embodiments, the emulsions contain one or more biosurfactants, one or more anionic surfactants, optionally, one or more nonionic surfactants, or mixtures thereof.

In a preferred embodiment, the compositions of the instant disclosure include a plurality of surfactants, wherein the plurality of surfactants include one or more biosurfactants and one or more surfactants other than the one or more biosurfactants. In further embodiments, the compositions of the instant disclosure include one or more biosurfactants, one or more anionic surfactants, and optionally, one or more nonionic surfactants.

In a preferred embodiment, the cosmetic composition includes one or more biosurfactants. In further embodiments, the cosmetic composition includes one or more biosurfactants and one or more additional surfactants different from the one or more biosurfactants selected from anionic surfactants, nonionic surfactants, amphoteric surfactants, or combinations thereof.

### Biosurfactant

Biosurfactants are amphiphilic molecules, for example, glycolipids (e.g., sophorolipids, rhamnolipids, cellobiose lipids, mannosylerythritol lipids and trehalose lipids), lipopeptides (e.g., surfactin, iturin, fengycin, arthrofactin and lichenysin), flavolipids, phospholipids (e.g., cardiolipins), fatty acid ester compounds, fatty acid ether compounds, and high molecular weight polymers such as lipoproteins, lipopolysaccharide-protein complexes, and polysaccharide-protein-fatty acid complexes.

Biosurfactants are environmentally friendly, biodegradable, and non-toxic and may be classified into high and low molecular weight biosurfactants. Low molecular weight biosurfactant efficiently lower surface and interfacial tension, and high molecular weight biosurfactants are more effective as emulsion-stabilizing agents. Examples of low molecular weight biosurfactants include glycolipids, such as rhamnolipids, sophorolipids, lipopeptidesm, and trehalolipids. These low molecular weight biosurfactants have hydrophilic heads comprised of sugar units linked glycosidically with hydrophobic non-polar parts. Examples of high molecular weight biosurfactants include polysaccharides, lipopolysaccharides, proteins, and lipoproteins. Polysaccharide-based biosurfactant can be classified into sorbitan esters, sucrose esters and glucose-based surfactants that include alkyl polyglycosides and fatty acid glucamides.

Nonlimiting examples of biosurfactants include liptopeptides such as surfactin; fatty acids and phospholipids, polymeric matrix biosurfactants; particulate biosurfactants; and bacterial biosurfactants composed of polysaccharides, proteins, lipopolysaccharides, lipoproteins or complex mixtures of these biopolymers.

Nonlimiting examples of commercially available biosurfactants includealkyl polyglycoside available under the trademark ECOSENSE®3000 from Dow Chemical®; D-glucopyranose, oligomeric, decyl octyl glycosides available under the trademark GLUCOPON®215 from BASF Corporation®; rhamnolipids available under the trademark REWOFERM® SL ONE from Evonik®; D-Glucitol, 1-deoxy-1-(methylamino)-, N-coco acyl derivatives available under the trademark GLUCOTAIN® from Clariant®; rhamnolipids from Jeneil Biotech®, and BioLoop® surfactants from Lankem® Ltd.

In one embodiment, the microbial biosurfactant is a glycolipid such as rhamnolipids (RLP), sophorolipids (SLP), trehalose lipid or mannosylerythritol lipid (MEL). The biosurfactants can be added in purified form or can be present in the microbe-based composition as a result of microbial growth. The biosurfactant may be a sophorolipid. In some embodiments, the biosurfactant can also be a lipopeptide, such as surfactin, and/or a rhamnolipid.

In some embodiments, a blend of biosurfactants is present. Preferably the blend comprises a rhamnolipid, and optionally one or both of a mannosylerythritol lipid, a surfactin or a sophorolipid. In a preferred embodiment, the microbe is a non-pathogenic strain of Pseudomonas. Preferably, the strain is a producer of rhamnolipid (RLP) biosurfactants.

Other microbial strains including, for example, other fungal strains capable of accumulating significant amounts of, for example, glycolipid-biosurfactants can be used in accordance with the subject invention. Biosurfactants useful according to the present invention include mannoprotein, beta-glucan and other metabolites that have bio-emulsifying and surface/interfacial tension-reducing properties.

In various embodiments, the one or more biosurfactants are selected from surfactin, iturin, fengycin, lichenysin, serrawettin, phospholipids, rhamnolipid, sophorolipid, trehalolipid, mannosylerythritol-lipids, cellobiolipids, lipoproteins, rubiwettins, trehalose, ornithin, pentasaccharide lipids, viscosin, bacitracin, lipopeptides, and combinations thereof. In one embodiment, the biosurfactants are selected from one or more glycolipids such as, for example, rhamnolipids, rhamnose-d-phospholipids, trehalose lipids, trehalose dimycolates, trehalose monomycolates, mannosylerythritol lipids, cellobiose lipids, ustilagic acid and/or sophorolipids.

In various embodiments, the biosurfactant has an anionic character, for example, sophorolipids, trehalolipid and rhamnolipids. Preferable are the mono-rhamnolipids and di-rhamnolipids. The preferred alkyl chain length is from C₈ to C₁₂. The alkyl chain may be saturated or unsaturated.

The term “rhamnolipids” includes compounds of the general formula (II) and salts thereof,

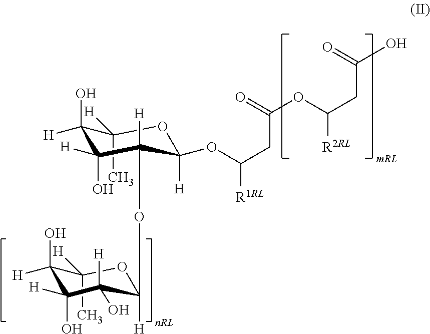

wherein,
mRL=2, 1 or 0,
nRL=1 or 0, R^{1RL} and R^{2RL}=are independently organic residues having 2 to 24, preferably 5 to 13 carbon atoms, in particular optionally branched, optionally substituted, particularly hydroxy-substituted, optionally unsaturated, in particular optionally mono-, bi- or tri-unsaturated alkyl residues, preferably those selected from the group consisting of pentenyl, heptenyl, nonenyl, undecenyl and tridecenyl and (CH₂)ₒ—CHs where o=1 to 23, preferably 4 to 12.

If nRL=1, the glycosidic bond between the two rhamnose units is preferably in the α-configuration. The optically active carbon atoms of the fatty acids are preferably present as R-enantiomers (e.g. I-3-{I-3-[2-O-(α-L-rhamnopyranosyl)-α-L-rhamnopyranosyl]oxydecanoyl}oxydecanoate).

The term “di-rhamnolipid” in the context of the present invention is understood to mean compounds of the general formula (II) or salts thereof, where nRL=1.

The term “mono-rhamnolipid” in the context of the present invention is understood to mean compounds of the general formula (II) or salts thereof, where nRL=0.

Distinct rhamnolipids are abbreviated according to the following nomenclature: “diRL-CXCY” are understood to mean di-rhamnolipids of the general formula (II), in which one of the residues R^{1RL} and R^{2RL}═(CH₂)ₒ—CH₃ where o=X−4 and the remaining residue R¹ or R²═(CH₂)ₒ—CH₃ where o=Y−4.

“monoRL-CXCY” are understood to mean mono-rhamnolipids of the general formula (II), in which one of the residues R^{1RL} and R^{2RL}═(CH·sub·₂)·sub·ₒ—CH·sub·₃ where o=X−4 and the remaining residue R^{1RL} or R^{2RL}═(CH₂)ₒ—CH₃ where o=Y−4. The nomenclature used therefore does not distinguish between “CXCY” and “CYCX”.

For rhamnolipids where mRL=0, monoRL-CX or diRL-CX is used accordingly.

If one of the abovementioned indices X and/or Y is provided with “:Z”, this signifies that the respective residue R^{1RL} and/or R^{2RL} is equal to an unbranched, unsubstituted hydrocarbon residue having X−3 or Y−3 carbon atoms having Z double bonds.

Methods for preparing the relevant rhamnolipids are disclosed, for example, in EP2786743 and EP2787065, which are incorporated herein by reference in their entirety. Rhamolipids can also be produced by fermentation of Pseudomonas, especially Pseudomonas aeruginosa, which are preferably non genetically modified cells, a technology already disclosed in the eighties, as documented e.g. in EP0282942 and DE4127908. Rhamnolipids produced in Pseudomonas aeruginosa cells which have been Improved for higher rhamnolipid titres by genetical modification can also be used in the context of the instant invention; such cells have for example been disclosed by Lei et al. in BIOTECHNOL LETT. 2020 June; 42(6):997-1002, which is incorporated herein by reference in its entirety. The biosurfactants, in particular glycolipid surfactants, can be produced e.g. as in EP 0 499 434, U.S. Pat. No. 7,985,722, WO 03/006146, JP 60 183032, DE 19648439, DE 19600743, JP 01 304034, CN 1337439, JP 2006 274233, KR 2004033376, JP 2006 083238, JP 2006 070231, WO 03/002700, FR 2740779, DE 2939519, U.S. Pat. No. 7,556,654, FR 2855752, EP 1445302, JP 2008 062179 and JP 2007 181789, which are incorporated herein by reference in their entirety.

Rhamnolipids produced by Pseudomonas aeruginosa are commercially available from Jeneil Biotech Inc., e.g. under the tradename ZONIX®, from Logos Technologies (technology acquired by Stepan), e.g. under the tradename NATSURFACT®, from Biotensidon GmbH, e.g. under the tradename RHAPYNAL®, from AGAE® technologies, e.g. under the name R90, R95, R95Md, R95Dd, from Locus Bio-Energy Solutions and from Shanghai Yusheng Industry Co. Ltd., e.g. under the tradename BIO-201 GLYCOLIPIDS®.

The total amount of the one or more biosurfactants in the cosmetic composition, if present, will vary but may be from about 0.1 to about 15 wt. %, based on the total weight of the cosmetic composition. In further embodiments, the total amount of the one or more biosurfactants in the cosmetic composition is from about 0.1 to about 10 wt. %, or about 0.1 to about 5 wt. %. In a further embodiment, the total amount of the one or more biosurfactants in the cosmetic composition is from about 0.5 to about 15 wt. %, about 0.5 to about 10 wt. %, or about 0.5 to about 5 wt. %, based on the total weight of the cosmetic composition. In yet a further embodiment, the total amount of the one or more biosurfactants in the cosmetic composition is from about 1 to about 15 wt. %, about 1 to about 10 wt. %, or about 1 to about 5 wt. %, based on the total weight of the cosmetic composition. In preferred embodiments, the total amount of the one or more biosurfactants in the cosmetic composition is from about 1 to about 8 wt. %, about 1 to about 6 wt. %, about 1 to about 5 wt. %, about 1 to about 4 wt. %, or about 1 to about 3 wt. %, based on the total weight of the cosmetic composition.

### Anionic Surfactants

Popular anionic surfactants known for their good detersive and foaming properties include sodium lauryl sulfate and sodium laureth ether sulfate, which may be included. In other embodiments, it can be preferable for the cosmetic composition to be free or essentially free from sulfate-based surfactants. In various embodiments, it can preferably to include gentler, non-sulfate anionic surfactants, such as one or more anionic surfactants selected from alkyl sulfonates, alkyl sulfosuccinates, alkyl sulfoacetates, acyl isethionates, alkoxylated monoacids, acyl amino acids such as acyl taurates, acyl glycinates, acyl glutamates, acyl sarcosinates, salts thereof, or mixtures thereof.

The total amount of the one or more anionic surfactants in the cosmetic composition will vary but can be in an amount from about 0.01 to about 10 wt. %, based on the total weight of the cosmetic composition. In further embodiments, the cosmetic composition can include from about 0.01 to about 8 wt. %, about 0.01 to about 6 wt. %, about 0.01 to about 5 wt. %, about 0.01 to about 3 wt. %, about 0.1 to about 10 wt. %, about 0.1 to about 8 wt. %, about 0.1 to about 6 wt. %, about 0.1 to about 5 wt. %, about 0.1 to about 3 wt. %, about 0.5 to about 10 wt. %, about 0.5 to about 8 wt. %, about 0.5 to about 6 wt. %, about 0.5 to about 5 wt. %, about 0.5 to about 3 wt. %, about 1 to about 10 wt. %, about 1 to about 8 wt. %, about 1 to about 6 wt. %, about 1 to about 5 wt. %, or about 1 to about 3 wt. % of the one or more anionic surfactants, based on the total weight of the cosmetic composition.

### (a) Acyl Isethionates

Non-limiting examples of useful acyl isethionates include those of formula (III) and (IV):

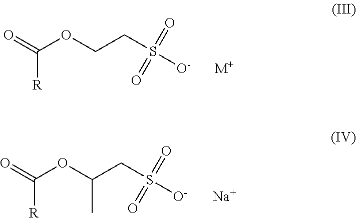

wherein R, R¹, R² and R³ are each independently selected from H or an alkyl chain having 1-24 carbon atoms, said chain being saturated or unsaturated, linear or branched, and X is COO⁻ or SO₃⁻. Although sodium is shown as the cation in formulae (III) and (IV), the cation for both formula (III) and formula (IV) may be an alkali metal ion such as sodium or potassium, ammonium ions, or alkanolammonium ions such as monoethanolammonium or triethanolammonium ions. Non-limiting examples of acyl isethionates include sodium isethionate, sodium cocoyl isethionate, sodium lauroyl methyl isethionate, and sodium cocoyl methyl isethionate. In some embodiments, a combination of sodium isethionate and sodium cocoyl isethionate are preferable.

### (b) Alkyl Sulfonates

Examples of alkyl sulfonates include alkyl aryl sulfonates, primary alkane disulfonates, alkene sulfonates, hydroxyalkane sulfonates, alkyl glyceryl ether sulfonates, alpha-olefinsulfonates, sulfonates of alkylphenolpolyglycol ethers, alkylbenzenesulfonates, phenvlalkanesulfonates, alpha-olefinsulfonates, olefin sulfonates, alkene sulfonates, hydroxyalkanesulfonates and disulfonates, secondary alkanesulfonates, paraffin sulfonates, ester sulfonates, sulfonated fatty acid glycerol esters, and alpha-sulfo fatty acid methyl esters including methyl ester sulfonate.

In some instances, an alkyl sulfonate of formula (V) is particularly useful.

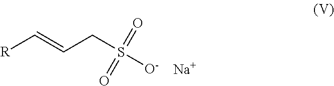

R is selected from H or alkyl chain that has 1-24 carbon atoms, preferably 6-24 carbon atoms, more preferably, 8 to 20 carbon atoms, said chain being saturated or unsaturated, linear or branched. Sodium is shown as the cation in the above formula (V) but the cation may be an alkali metal ion such as sodium or potassium, ammonium ions, or alkanolammonium ions such as monoethanolammonium or triethanolammonium ions. In some instances, the alkyl sulfonate(s) are selected from C₈-C₁₆ alkyl benzene sulfonates, C₁₀-C₂₀ paraffin sulfonates, C₁₀-C₂₄ olefin sulfonates, salts thereof, and mixtures thereof. C₁₀-C₂₄ olefin sulfonates may be particularly preferred. A non-limiting example of a C₁₀-C₂₄ olefin sulfonate that can be used in the instant compositions is sodium C₁₄-C₁₆ olefin sulfonate.

### (c) Alkyl Sulfosuccinates

Non-limiting examples of useful sulfosuccinates include those of formula (VI):

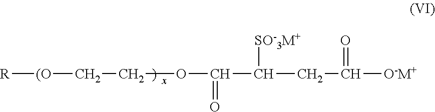

wherein R is a straight or branched chain alkyl or alkenyl group having 10 to 22 carbon atoms, preferably 10 to 20 carbon atoms, X is a number that represents the average degree of ethoxylation and can range from 0 to about 5, preferably from 0 to about 4, and most preferably from about 2 to about 3.5, and M and M′ are monovalent cations which can be the same or different from each other. Preferred cations are alkali metal ions such as sodium or potassium, ammonium ions, or alkanolammonium ions such as monoethanolammonium or triethanolammonium ions.

Non-limiting examples of alkyl sulfosuccinates salts include disodium oleamido MIPA sulfosuccinate, disodium oleamido MEA sulfosuccinate, disodium lauryl sulfosuccinate, disodium laureth sulfosuccinate, diammonium lauryl sulfosuccinate, diammonium laureth sulfosuccinate, dioctyl sodium sulfosuccinate, disodium oleamide MEA sulfosuccinate, sodium dialkyl sulfosuccinate, and a mixture thereof. In some instances, disodium laureth sulfosuccinate is particularly preferred.

### (d) Alkyl Sulfoacetates

Non-limiting examples of alkyl sulfacetates includes, for example, alkyl sulfoacetates such as C4-C18 fatty alcohol sulfoacetates and/or salts thereof. A particularly preferred sulfoacetate salt is sodium lauryl sulfoacetate. Useful cations for the salts include alkali metal ions such as sodium or potassium, ammonium ions, or alkanolammonium ions such as monoethanolammonium or triethanolammonium ions.

### (e) Alkoxylated Monoacids

Non-limiting examples of alkoxylated monoacids include compounds corresponding to formula (VII):

RO[CH2O]u[(CH2)xCH(R′)(CH2)y(CH2)zO]v[CH2CH2O]wCH2COOH (VII)
wherein:
R is a hydrocarbon radical containing from about 6 to about 40 carbon atoms;
u, v and w, independently of one another, represent numbers of from 0 to 60;
x, y and z, independently of one another, represent numbers of from 0 to 13;
R′ represents hydrogen, alkyl, and
the sum of x+y+z>0;

Compounds corresponding to formula (VII) can be obtained by alkoxylation of alcohols ROH with ethylene oxide as the sole alkoxide or with several alkoxides and subsequent oxidation. The numbers u, v, and w each represent the degree of alkoxylation. Whereas, on a molecular level, the numbers u, v and w and the total degree of alkoxylation can only be integers, including zero, on a macroscopic level they are mean values in the form of broken numbers.

In formula (VII), R is linear or branched, acyclic or cyclic, saturated or unsaturated, aliphatic or aromatic, substituted or unsubstituted. Typically, R is a linear or branched, acyclic C₆-C₄₀ alkyl or alkenyl group or a C₁-C₄₀ alkyl phenyl group, more typically a C₈-C₂₂ alkyl or alkenyl group or a C₄-C₁₈ alkyl phenyl group, and even more typically a C₁₂-C₁₈ alkyl group or alkenyl group or a C₆-C₁₆ alkyl phenyl group; u, v, w, independently of one another, is typically a number from 2 to 20, more typically a number from 3 to 17 and most typically a number from 5 to 15; x, y, z, independently of one another, is typically a number from 2 to 13, more typically a number from 1 to 10 and most typically a number from 0 to 8.

Suitable alkoxylated monoacids include, but are not limited to: Butoxynol-5 Carboxylic Acid, Butoxynol-19 Carboxylic Acid, Capryleth-4 Carboxylic Acid, Capryleth-6 Carboxylic Acid, Capryleth-9 Carboxylic Acid, Ceteareth-25 Carboxylic Acid, Coceth-7 Carboxylic Acid, C₉-C₁₁ Pareth-6 Carboxylic Acid, C₁₁-C₁₅ Pareth-7 Carboxylic Acid, C₁₂-C₁₃ Pareth-5 Carboxylic Acid, C₁₂-C₁₃ Pareth-8 Carboxylic Acid, C₁₂-C₁₃ Pareth-12 Carboxylic Acid, C₁₂-C₁₅ Pareth-7 Carboxylic Acid, C₁₂-C₁₅ Pareth-8 Carboxylic Acid, C₁₄-C₁₅ Pareth-8 Carboxylic Acid, Deceth-7 Carboxylic Acid, Laureth-3 Carboxylic Acid, Laureth-4 Carboxylic Acid, Laureth-5 Carboxylic Acid, Laureth-6 Carboxylic Acid, Laureth-8 Carboxylic Acid, Laureth-10 Carboxylic Acid, Laureth-11 Carboxylic Acid, Laureth-12 Carboxylic Acid, Laureth-13 Carboxylic Acid, Laureth-14 Carboxylic Acid, Laureth-17 Carboxylic Acid, PPG-6-Laureth-6 Carboxylic Acid, PPG-8-Steareth-7 Carboxylic Acid, Myreth-3 Carboxylic Acid, Myreth-5 Carboxylic Acid, Nonoxynol-5 Carboxylic Acid, Nonoxynol-8 Carboxylic Acid, Nonoxynol-10 Carboxylic Acid, Octeth-3 Carboxylic Acid, Octoxynol-20 Carboxylic Acid, Oleth-3 Carboxylic Acid, Oleth-6 Carboxylic Acid, Oleth-10 Carboxylic Acid, PPG-3-Deceth-2 Carboxylic Acid, Capryleth-2 Carboxylic Acid, Ceteth-13 Carboxylic Acid, Deceth-2 Carboxylic Acid, Hexeth-4 Carboxylic Acid, Isosteareth-6 Carboxylic Acid, Isosteareth-11 Carboxylic Acid, Trudeceth-3 Carboxylic Acid, Trideceth-6 Carboxylic Acid, Trideceth-8 Carboxylic Acid, Trideceth-12 Carboxylic Acid, Trideceth-3 Carboxylic Acid, Trideceth-4 Carboxylic Acid, Trideceth-7 Carboxylic Acid, Trideceth-15 Carboxylic Acid, Trideceth-19 Carboxylic Acid, Undeceth-5 Carboxylic Acid and mixtures thereof. In some cases, preferred ethoxylated acids include Oleth-10 Carboxylic Acid, Laureth-5 Carboxylic Acid, Laureth-11 Carboxylic Acid, and a mixture thereof.

### (f) Acyl Amino Acids

Acyl amino acids that may be used include, but are not limited to, amino acid surfactants based on alanine, arginine, aspartic acid, glutamic acid, glycine, isoleucine, leucine, lysine, phenylalanine, serine, tyrosine, valine, sarcosine, threonine, and taurine. The most common cation associated with the acyl amino acid can be sodium or potassium. Alternatively, the cation can be an organic salt such as triethanolamine (TEA) or a metal salt. Non-limiting examples of acyl amino acids include those of formula (VIII):

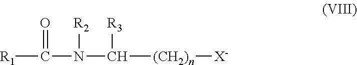

wherein R, R¹, R² and R³ are each independently selected from H or an alkyl chain having 1-24 carbon atoms, said chain being saturated or unsaturated, linear or branched, and X is COO⁻ or SO₃⁻.

### (g) Acyl Taurates

Non-limiting examples of acyl taurates include those of formula (IX):

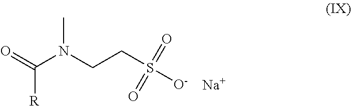

wherein R, R¹, R² and R³ are each independently selected from H or an alkyl chain having 1-24 carbon atoms, or from 6-20 carbon atoms, or from 8 to 16 carbon atoms, said chain being saturated or unsaturated, linear or branched, and X is COO⁻ or SO₃⁻. Non-limiting examples of acyl taurate salts include sodium cocoyl taurate, sodium methyl cocoyl taurate, sodium lauroyl taurate, and sodium methyl lauroyl taurate.

### (h) Acyl Glycinates

Non-limiting examples of acyl glycinates include those of formula (X):

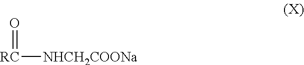

wherein R is an alkyl chain of 8 to 16 carbon atoms. Although sodium is shown as the cation in the above formula (X), the cation may be an alkali metal ion such as sodium or potassium, ammonium ions, or alkanolammonium ions such as monoethanolammonium or triethanolammonium ions. Non-limiting examples of acyl glycinates include sodium cocoyl glycinate, sodium lauroyl glycinate, sodium myristoyl glycinate, potassium lauroyl glycinate, and potassium cocoyl glycinate, and in particular sodium cocoyl glycinate.

### (i) Acyl Glutamates

Non-limiting examples of acyl glutamates include those of formula (XI):

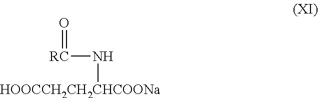

wherein R is an alkyl chain of 8 to 16 carbon atoms. Sodium is shown as the cation in the above formula (XI) but the cation may be an alkali metal ion such as sodium or potassium, ammonium ions, or alkanolammonium ions such as monoethanolammonium or triethanolammonium ions. Non-limiting examples of acyl gluatamtes include dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, sodium undecylenoyl glutamate, triethanolamine mono-cocoyl glutamate, triethanolamine lauroylglutamate, and disodium cocoyl glutamate. In some cases, sodium stearoyl glutamate is particularly preferred.

### (j) Acyl Sarcosinates:

Non-limiting examples of acyl sarcosinates include potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate, and ammonium lauroyl sarcosinate.

The total amount of the one or more anionic surfactants in the cosmetic composition, if present, will vary but may be from about 0.01 to about 6 wt. %, based on the total weight of the cosmetic composition. In further embodiments, the total amount of the one or more anionic surfactants in the cosmetic composition, if present, is from about 0.01 to about 5 wt. %, about 0.01 to about 3 wt. %, about 0.1 to about 6 wt. %, about 0.1 to about 5 wt. %, about 0.1 to about 3 wt. %, about 0.5 to about 6 wt. %, about 0.5 to about 5 wt. %, about 0.5 to about 3 wt. %, based on the total weight of the cosmetic composition.

### Amphoteric Surfactants

The multiphase cleansing compositions may optionally include one or more amphoteric surfactants. Nonlimiting examples of amphoteric surfactants include betaines, alkyl amphoacetates and alkyl amphodiacetates, alkyl sulltaines, alkyl amphopropionates, and combinations thereof.

Nonlimiting examples of betaine surfactants include coco-betaine, cocamidopropyl betaine, lauryl betaine, laurylhydroxy sulfobetaine, lauryldimethyl betaine, cocamidopropyl hydroxysultaine, behenyl betaine, capryl/capramidopropyl betaine, lauryl hydroxysultaine, stearyl betaine, or mixtures thereof.

Nonlimiting examples of alkyl amphoacetates and alkyl amphodiacetates include (C8-C20)alkylamphoacetates and (C8-C20)alkylamphodiacetates, such as disodium cocoamphodiacetate, disodium lauroamphodiacetate, disodium caprylamphodiacetate, disodium caprylamphodiacetate, disodium cocoamphodipropionate, disodium lauroamphodipropionate, disodium caprylampho-dipropionate, disodium caprylomphodipropionate, lauroamphodipropionic acid, cocoamphodipropionic acid, and combinations thereof.

The total amount of the one or more amphoteric surfactants in the cosmetic composition will vary but can be from about 0.01 to about 10 wt. %, based on the total weight of the final cosmetic composition. In further embodiments, the cosmetic composition can include from about 0.01 to about 8 wt. %, about 0.01 to about 6 wt. %, about 0.01 to about 5 wt. %, about 0.01 to about 3 wt. %, about 0.1 to about 10 wt. %, about 0.1 to about 8 wt. %, about 0.1 to about 6 wt. %, about 0.1 to about 5 wt. %, about 0.1 to about 3 wt. %, about 0.5 to about 10 wt. %, about 0.5 to about 8 wt. %, about 0.5 to about 6 wt. %, about 0.5 to about 5 wt. %, about 0.5 to about 3 wt. %, about 1 to about 10 wt. %, about 1 to about 8 wt. %, about 1 to about 6 wt. %, about 1 to about 5 wt. %, or about 1 to about 3 wt. % of the one or more amphoteric surfactants, based on the total weight of the cosmetic composition.

### Nonionic Surfactants

The multiphase cleansing composition may optionally include one or more nonionic surfactants. Nonlimiting examples of nonionic surfactants include alkoxylated fatty alcohols, alkoxylated polyol esters, alkoxylated glycerides, glucosides, alkanolamides, sorbitan derivatives, or combinations thereof.

Nonlimiting examples of alkoxylated fatty alcohols include laureth-3, laureth-4, laureth-7, laureth-9, laureth-12, laureth-23, ceteth-10, steareth-10, steareth-2, steareth-100, beheneth-5, beheneth-5, beheneth-10, oleth-10, Pareth alcohols, trideceth-10, trideceth-12, C12-13 pareth-3, C12-13 pareth-23, C11-15 pareth-7, PEG hydrogenated castore oil, PEG-75 lanolin, polysorbate-80, polysobate-20, PPG-5 ceteth-20, PEG-55 Propylene Glycol Oleate, glycereth-26 (PEG-26 Glyceryl Ether), PEG 120 methyl glucose dioleate, PEG 120 methyl glucose trioleate, PEG 150 pentaerythrityl tetrastearate, and mixtures thereof.

Nonlimiting examples of alkoxylatecd polyol esters include the adducts of ethylene oxide with esters of lauric acid, palmitic acid, stearic acid or behenic acid, and mixtures thereof, especially those containing from 9 to 100 oxyethylene groups, such as PEG-9 to PEG-50 laurate (as the INCI names: PEG-9 laurate to PEG-50 laurate); PEG-9 to PEG-50 palmitate (as the INCI names: PEG-9 palmitate to PEG-50 palmitate); PEG-9 to PEG-50 stearate (as the INCI names: PEG-9 stearate to PEG-50 stearate); PEG-9 to PEG-50 palmitostearate; PEG-9 to PEG-50 behenate (as the INCI names: PEG-9 behenate to PEG-50 behenate); polyethylene glycol 100 EO monostearate (INCI name: PEG-100 stearate); and mixtures thereof.

Nonlimiting examples of alkoxylated glycerides include PEG-6 almond glycerides, PEG-20 almond glycerides, PEG-35 almond glycerides, PEG-60 almond glycerides, PEG-192 apricot kernel glycerides, PEG-11 avocado glycerides, PEG-14 avocado glycerides, PEG-11 babassu glycerides, PEG-42 babassu glycerides, PEG-4 caprylic/capric glycerides, PEG-6 caprylic/capric glycerides, PEG-7 caprylic/capric glycerides, PEG-8 caprylic/capric glycerides, PEG-11 cocoa butter glycerides, PEG-75 cocoa butter glycerides, PEG-7 cocoglycerides, PEG-9 cocoglycerides, PEG-20 corn glycerides, PEG-60 corn glycerides, PEG-20 evening primrose glycerides, PEG-60 evening primrose glycerides, PEG-5 hydrogenated corn glycerides, PEG-8 hydrogenated fish glycerides, PEG-20 hydrogenated palm glycerides, PEG-6 hydrogenated palm/palm kernel glyceride, PEG-16 macadamia glycerides, PEG-70 mango glycerides, PEG-13 mink glycerides, PEG-25 moringa glycerides, PEG-42 mushroom glycerides, PEG-2 olive glycerides, PEG-6 olive glycerides, PEG-7 olive glycerides, PEG-10 olive glycerides, PEG-40 olive glycerides, PEG-18 palm glycerides, PEG-12 palm kernel glycerides, PEG-45 palm kernel glycerides, PEG-60 Passiflora edulis seed glycerides, PEG-60 Passiflora incarnata seed glycerides, PEG-45 safflower glycerides, PEG-60 shea butter glycerides, PEG-75 shea butter glycerides, PEG-75 shorea butter glycerides, PEG-35 soy glycerides, PEG-75 soy glycerides, PEG-2 sunflower glycerides, PEG-7 sunflower glycerides, PEG-10 sunflower glycerides, PEG-13 sunflower glycerides, PEG-5 tsubakiate glycerides, PEG-10 tsubakiate glycerides, PEG-20 tsubakiate glycerides, PEG-60 tsubakiate glycerides, sodium PEG-8 palm glycerides carboxylate, or mixtures thereof.

Nonlimiting examples of glucosides (also known as “alkyl polyglucosides”) include lauryl glucoside, octyl glucoside, decyl glucoside, coco glucoside, caprylyl/capryl glucoside, sodium lauryl glucose carboxylate, or combinations thereoif.

Nonlimiting examples of alkanolamides include cocamide MIPA, cocamide DEA, cocamide MEA, cocamide DIPA, and mixtures thereof.

Nonlimiting examples of sorbitan derivatives include polysorbates, for example, polysorbate-20 (POE(20) sorbitan monolaurate), polysorbate-21 (POE(4) sorbitan monolaurate), polysorbate-40 (POE(20) sorbitan monopalmitate), polysorbate-60 (POE(20) sorbitan monostearate), polysorbate-61 (POE(4) sorbitan monostearate), polysorbate-65 (POE(20) sorbitan tristearate), polysorbate-80 (POE(20)sorbitan monooleate), polysorbate-81 (POE(4) sorbitan monooleate), polysorbate 85 (POE(20) Sorbitan Trioleate), sorbitan isostearate, sorbitan monolaurate, sorbitan monooleate, sorbitan monopalmitate, sorbitan monostearate, sorbitan sesquioleate, sorbitan trioleate sorbitan tristearate, or mixtures thereof.

The total amount of the one or more nonionic surfactants in the cosmetic composition will vary but can be from about 0.01 to about 10 wt. %, based on the total weight of the final cosmetic composition. In further embodiments, the cosmetic composition can include from about 0.01 to about 8 wt. %, about 0.01 to about 6 wt. %, about 0.01 to about 5 wt. %, about 0.01 to about 3 wt. %, about 0.1 to about 10 wt. %, about 0.1 to about 8 wt. %, about 0.1 to about 6 wt. %, about 0.1 to about 5 wt. %, about 0.1 to about 3 wt. %, about 0.5 to about 10 wt. %, about 0.5 to about 8 wt. %, about 0.5 to about 6 wt. %, about 0.5 to about 5 wt. %, about 0.5 to about 3 wt. %, about 1 to about 10 wt. %, about 1 to about 8 wt. %, about 1 to about 6 wt. %, about 1 to about 5 wt. %, or about 1 to about 3 wt. % of the one or more nonionic surfactants, based on the total weight of the cosmetic composition.

### Cationic Surfactants

Non-limiting examples of cationic surfactants include cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyl dimethylamine, behenamidopropyl dimethylamine, behenamidopropyl diethylamine, behenamidoethyl diethylamine, behenamidoethyl dimethylamine, arachidamidopropyl dimethylamine, arachidamidopropyl diethylamine, arachidamidoethyl diethylamine, arachidamidoethyl dimethylamine, brassicamidopropyl dimethylamine, lauramidopropyl dimethylamine, myristamidopropyl dimethylamine, dilinoleamidopropyl dimethylamine, palmitamidopropyl dimethylamine, and mixtures thereof.

The one or more cationic surfactants may be selected from quaternary ammonium compounds, fatty dialkylamines, or mixtures thereof.

Nonlimiting examples of quaternary ammonium compounds include cetrimonium chloride, steartrimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, and combinations thereof.

Nonlimiting examples of fatty dialkylamines include oleamidopropyl dimethylamine, linoleamidopropyl dimethylamine, isostearamidopropyl dimethylamine, oleyl hydroxyethyl imidazoline, stearamidopropyl dimethylamine, behenamidopropyl dimethylamine, behenamidopropyl diethylamine, behenamidoethyl diethylamine, behenamidoethyl dimethylamine, arachidamidopropyl dimethylamine, arachidamidopropyl diethylamine, arachidamidoethyl diethylamine, arachidamidoethyl dimethylamine, brassicamidopropyl dimethylamine, lauramidopropyl dimethylamine, myristamidopropyl dimethylamine, dilinoleamidopropyl dimethylamine, palmitamidopropyl dimethylamine, salts thereof, and combinations thereof.

In various embodiments, the one or more cationic surfactants are preferably selected from cetrimonium chloride, behentrimonium chloride, behentrimonium methosulfate, stearamidopropyl dimethylamine, brassicamidopropyl dimethylamine or a mixture thereof.

The total amount of the one or more cationic surfactants in the cosmetic composition will vary but can be from about 0.01 to about 10 wt. %, based on the total weight of the cosmetic composition. In further embodiments, the cosmetic composition includes from about 0.01 to about 8 wt. %, about 0.01 to about 6 wt. %, about 0.01 to about 5 wt. %, about 0.01 to about 3 wt. %, about 0.1 to about 10 wt. %, about 0.1 to about 8 wt. %, about 0.1 to about 6 wt. %, about 0.1 to about 5 wt. %, about 0.1 to about 3 wt. %, about 0.5 to about 10 wt. %, about 0.5 to about 8 wt. %, about 0.5 to about 6 wt. %, about 0.5 to about 5 wt. %, about 0.5 to about 3 wt. %, about 1 to about 10 wt. %, about 1 to about 8 wt. %, about 1 to about 6 wt. %, about 1 to about 5 wt. %, or about 1 to about 3 wt. % of the one or more cationic surfactants, based on the total weight of the cosmetic composition.

### (e) Water

The total amount of water in the cosmetic composition will vary but can be from about 30 to about 90 wt. %, based on the total weight of the mineral cosmetic. In further embodiments, the total amount of water may be from about 30 to about 80 wt. %, about 30 to about 70 wt. %, about 30 to about 60 wt. %, about 30 to about 50 wt. %, about 40 to about 90 wt. %, about 40 to about 80 wt. %, about 40 to about 70 wt. %, about 40 to about 60 wt. %, about 40 to about 50 wt. %, about 50 to about 90 wt. %, about 50 to about 80 wt. %, about 50 to about 70 wt. %, about 50 to about 60 wt. %, about 60 to about 90 wt. %, about 60 to about 80 wt. %, about 50 to about 70 wt. %, based on the total weight of the cosmetic composition.

### Fatty Alcohols

The cosmetic composition may optionally include one or more fatty alcohols. Fatty alcohols typically include monohydric alcohols having 8-22 carbon atoms although longer chain alcohols in excess of 30 carbons may be used. The fatty alcohols may be saturated or unsaturated. The fatty alcohols may be straight or branched. In particular, the oil phase may comprise straight chain, saturated fatty alcohol with a terminal hydroxyl. Suitable fatty alcohols include decyl alcohol, lauryl alcohol, myristyl alcohol, cetyl alcohol, stearyl alcohol, isostearyl alcohol, cetearyl alcohol, icosyl alcohol, behenyl alcohol.

The total amount of one or more fatty alcohols, if present, will vary but can be from about 0.1 to about 10 wt. %, based on the total weight of the cosmetic composition. In further embodiments, the total amount of one or more fatty alcohols can be from about 0.1 to about 8 wt. %, about 0.1 to about 5 wt. %, about 0.5 to about 10 wt. %, about 0.5 to about 8 wt. %, about 0.5 to about 5 wt. %, about 1 to about 10 wt. %, about 1 to about 8 wt. %, about 1 to about 5 wt. %, about 2 to about 8 wt. %, about 5 to about 10 wt. %, or about 5 to about 15 wt. %, based on the total weight of the cosmetic compositions.

### Water Soluble Solvent

The cosmetic composition can optionally include one or more water soluble solvents. The term “water soluble solvent” is interchangeable with the terms “water soluble organic solvent” and “water-miscible solvent” and means a compound that is liquid at 25° C. and at atmospheric pressure (760 mmHg), and it has a solubility of at least 50% in water under these conditions. In some cases, the water-soluble solvents have a solubility of at least 60%, 70%, 80%, or 90%. Non-limiting examples of water-soluble solvents include, for example, organic solvents selected from glycerin, mono-alcohols (for example C₂₋₈, or C₂₋₄ alcohols), polyols (polyhydric alcohols), glycols, and a mixture thereof.

Nonlimiting examples of water-soluble organic solvents. Non-limiting examples of water-soluble organic solvents include, for example, organic solvents selected from alcohols (for example C₂₋₆ or C₂₋₄ alcohols), polyols (polyhydric alcohols), glycols, and a mixture thereof. Nonlimiting examples of monoalcohols and polyols include ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, and propane diol.

Further non-limiting examples of water soluble organic solvents include alkanediols (polyhydric alcohols) such as 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

Polyhydric alcohols are useful. Examples of polyhydric alcohols include ethylene glycol, diethylene glycol, triethylene glycol, propylene glycol, dipropylene glycol, tripropylene glycol, 1,3-butanediol, 2,3-butanediol, 1,4-butanediol, 3-methyl-1, 3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a mixture thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, and a mixture thereof. In a preferred embodiment, the composition include one or more glycols selected from propylene glycol, butylene glycol, pentylene glycol, hexylene glycol, caprylyl glycol, dipropylene glycol, and mixtures thereof.

The total amount of the one or more water soluble solvents in the cosmetic composition, if present, will vary but can be from about 0.1 to about 20 wt. %, based on the total weight of the cosmetic composition. In further embodiments, the cosmetic composition may include from about 0.1 to about 15 wt. %, about 0.1 to about 10 wt. %, about 0.1 to about 5 wt. %, about 1 to about 20 wt. %, about 1 to about 15 wt. %, about 1 to about 10 wt. %, or about 1 to about 5 wt. % of one or more water soluble solvents, based on the total weight of the cosmetic composition.

### Vitamins

The cosmetic composition may optionally include one or more skin vitamins. “Vitamins” means vitamins, pro-vitamins, and their salts, isomers and derivatives. Non-limiting examples of suitable vitamins include vitamin B compounds (including B1 compounds, B2 compounds, B3 compounds such as niacinamide, niacinnicotinic acid, tocopheryl nicotinate, C1-C18 nicotinic acid esters, and nicotinyl alcohol; B5 compounds, such as panthenol or “pro-B5”, pantothenic acid, pantothenyl; B6 compounds, such as pyroxidine, pyridoxal, pyridoxamine; carnitine, thiamine, riboflavin); vitamin A compounds, and all natural and/or synthetic analogs of Vitamin A, including retinoids, retinol, retinyl acetate, retinyl palmitate, retinoic acid, retinaldehyde, retinyl propionate, carotenoids (pro-vitamin A), and other compounds which possess the biological activity of Vitamin A; vitamin D compounds; vitamin K compounds; vitamin E compounds, or tocopherol, including tocopherol sorbate, tocopherol acetate, other esters of tocopherol and tocopheryl compounds; vitamin C compounds, including ascorbate, ascorbyl esters of fatty acids, and ascorbic acid derivatives, for example, ascorbyl phosphates such as magnesium ascorbyl phosphate and sodium ascorbyl phosphate, ascorbyl glucoside, and ascorbyl sorbate; and vitamin F compounds, such as saturated and/or unsaturated fatty acids. In one embodiment, the composition comprises a vitamin selected from the group consisting of vitamin B compounds, vitamin C compounds, vitamin E compounds and mixtures thereof. Alternatively, the vitamin is selected from the group consisting of niacinamide, tocopheryl nicotinate, pyroxidine, panthenol, vitamin E, vitamin E acetate, ascorbyl phosphates, ascorbyl glucoside, and mixtures thereof.

The total amount of one or more vitamins, if present, will vary but can be from about 0.01 to about 8 wt. %, based on the total weight of the cosmetic composition. In further embodiments, the cosmetic composition may include from about 0.01 to about 5 wt. %, about 0.01 to about 3 wt. %, about 0.1 to about 8 wt. %, about 0.1 to about 5 wt. % or about 0.1 to about 3 wt. % of one or more vitamins, based on the total weight of the mineral cosmetic.

### Antioxidants

The cosmetic compositions may optionally include one or more antioxidants. Suitable antioxidants include, but are not limited to, water-soluble antioxidants such as sulfhydryl compounds and their derivatives (e.g., sodium metabisulfite and N-acetyl-cysteine), lipoic acid and dihydrolipoic acid, resveratrol, lactoferrin, and ascorbic acid and ascorbic acid derivatives (e.g., ascorbyl palmitate, ascorbyl polypeptide and ascorbyl phosphate).

Oil-soluble antioxidants suitable for use in the compositions of this invention include, but are not limited to, butylated hydroxytoluene, retinoids (e.g., retinol and retinyl palmitate), tocopherols (e.g., tocopherol acetate), sodium tocopheryl phosphate, tocotrienols, alkylresorcinols, curcurmin and its derivatives and ubiquinone. Natural extracts containing antioxidants suitable for use in the compositions of this invention, include, but not limited to, extracts containing flavonoids and isoflavonoids and their derivatives (e.g., genistein and diadzein), extracts containing resveratrol and the like. Examples of such natural extracts include grape seed, green tea, pine bark, Phyllanthus emblica and propolis.

The total amount of one or more antioxidants, if present, will vary, but can be from about 0.1 to about 8 wt. %, based on the total weight of the cosmetic composition. In further embodiments, the cosmetic composition may include from about 0.01 to about 5 wt. %, about 0.01 to about 3 wt. %, about 0.1 to about 8 wt. %, about 0.1 to about 5 wt. %, or about 0.1 to about 3 wt. % of one or more antioxidants, based on the total weight of the cosmetic composition.

### Miscellaneous Ingredients

The cosmetic compositions may optionally include or exclude (or are essentially free from) one or more miscellaneous ingredients. Miscellaneous ingredients are ingredients that are compatible with the compositions and do not disrupt or materially affect the basic and novel properties of the compositions. Nonlimiting examples of miscellaneous ingredients include preservatives, fragrances, pH adjusters, salts, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, etc. In various embodiments, the miscellaneous ingredients are chosen from preservatives, fragrances, pH adjusters, salts, chelating agents, buffers, composition colorants, and mixtures thereof. In the context of the instant disclosure, a “composition colorant” is a compound that colors the composition but does not have an appreciable coloring effect on hair. In other words, the composition colorant is included to provide a coloring to the composition for aesthetic appeal but is not intended to impart coloring properties to hair. Styling gels, for example, can be found in a variety of different colors (e.g., light blue, light pink, etc.) yet application of the styling gel to hair does not visibly change the color of the hair.

The total amount of the one or more miscellaneous ingredients in the cosmetic composition, if present, will vary but may be in an amount from about 0.1 to about 15 wt. %, based on the total weight of the cosmetic composition. In further embodiments, the cosmetic composition includes about 0.1 to about 12 wt. %, about 0.1 to about 10 wt. %, about 0.1 to about 5 wt. %, about 0.5 to about 15 wt. %, about 0.5 to about 12 wt. %, about 0.5 to about 10 wt. %, about 0.5 to about 8 wt. %, about 0.5 to about 5 wt. %, about 1 to about 15 wt. %, about 1 to about 12 wt. %, about 1 to about 10 wt. %, about 1 to about 8 wt. %, about 1 to about 5 wt. %, about 2 to about 15 wt. %, about 2 to about 12 wt. %, about 2 to about 10 wt. %, about 2 to about 8 wt. %, or about 2 to about 5 wt. % of one or more miscellaneous ingredients, based on the total weight of the cosmetic composition.

### Aqueous Phase

The cosmetic compositions are in the form of an oil-in-water emulsions having an aqueous phase and an oil phase. The aqueous phase may constitute about 10 to about 90 wt. % of the cosmetic composition, based on the total weight of the cosmetic composition. In further embodiments, the aqueous phase may constitute about 20 to about 80 wt. %, about 30 to about 70 wt. %, about 40 to about 60 wt. %, about 10 to about 40 wt. %, about 20 to about 50 wt. %, about 30 to about 60 wt. %, about 40 to about 80 wt. %, about 50 to about 90 wt. %, or about 60 to about 90 wt. %, based on the total weight of the cosmetic composition.

### Oil Phase

The oil phase may constitute about 10 to about 90 wt. % of the cosmetic composition, based on the total weight of the cosmetic composition. In further embodiments, the oil phase may constitute about 20 to about 80 wt. %, about 30 to about 70 wt. %, about 40 to about 60 wt. %, about 10 to about 40 wt. %, about 20 to about 50 wt. %, about 30 to about 60 wt. %, about 40 to about 80 wt. %, about 50 to about 90 wt. %, or about 60 to about 90 wt. %, based on the total weight of the cosmetic composition.

### Droplet Size

In various embodiments, the average droplet size of the oil phase in the emulsions forming the cosmetic compositions is from about 10 nm to about 2 μm, about 10 nm to about 1.5 μm, about 10 nm to about 1 μm, about 10 nm to about 800 nm, about 10 nm to about 600 nm, about 10 nm to about 500 nm, about 10 nm to about 250 nm, about 50 nm to about 2 μm, about 50 nm to about 1.5 μm, about 50 nm to about 1 μm, about 50 nm to about 800 nm, about 50 nm to about 600 nm, about 50 nm to about 500 nm, about 50 nm to about 250 nm, about 100 nm to about 2 μm, about 100 nm to about 1.5 μm, about 100 nm to about 1 μm, about 100 nm to about 800 nm, about 100 nm to about 600 nm, about 100 nm to about 500 nm, about 100 nm to about 300 nm, about 150 nm to about 500 nm, about 150 nm to about 400 nm, about 200 nm to about 500 nm, or about 200 nm to about 300 nm.

Droplet size can be determined using Brookhaven Dynamic Light Scattering (DLS). DLS is a technique used to determine droplet size in a colloidal system or emulsion. When the samples are illuminated with a monochromatic laser beam, the particles in the samples undergo Brownian motion, causing fluctuations in scattered light intensity. The scattered light is then collected at various angles, and the autocorrelation function of these intensity fluctuations is analyzed. The analysis provides information about the rate of diffusion of the particles, and from this, the size distribution is inferred using mathematical models. Brookhaven's DLS instruments use advanced algorithms to accurately interpret the data, offering insights into the dynamic behavior and size characteristics of particles ranging from a few nanometers to several micrometers in a liquid medium.

### EMBODIMENTS

In a preferred embodiment, the cosmetic composition comprises, consists essentially of, or consists of:
(a) about 0.1 to about 8 wt. %, preferably about 0.1 to about 5 wt. %, more preferably about 0.5 to about 3 wt. % of a hydrophobic polymer, which is a reaction product of a natural or food-derived oil and a methacrylate or acrylate polymer, wherein preferably,
   the hydrophobic polymer is a reaction product of: (a)(i) a natural or food-derived oil selected from linseed oil, sunflower oil, tung oil, fish oil, cottonseed oil, soybean oil, or combinations thereof, preferably linseed oil and (a)(ii) a polymer derived from monomers selected from isobutyl methacrylate, methyl methacrylate, ethyl methacrylate, n-butyl methacrylate, and combinations thereof, preferably isobutyl methacrylate polymer, wherein even more preferably, the hydrophobic polymer is the reaction product of linseed oil and poly(isobutyl methacrylate);
(b) one or more solvents capable of solubilizing the reaction product of (a), wherein preferably,
   the one or more solvents capable of solubilizing the reaction product of (a) have a dispersion component (D), a polar component (P), and a hydrogen bonding component (H), and a distance (Ra) of less than or equal to 13.4 MPa^{0.5} per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
D₁ is 16.8 MPa^{0.5},
P₁ is 4.8 MPa^{0.5}, and
H₁ is 13.0 MPa^{0.5}, wherein more preferably,
the one or more solvents capable of solubilizing the reaction product of (a) have a dispersion component (D), a polar component (P), and a hydrogen bonding component (H), and a distance (Ra) of less than or equal to 9.9 MPa^{0.5} per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
D₁ is 16.4 MPa^{0.5},
P₁ is 5.0 MPa^{0.5}, and
H₁ is 11.7 MPa^{0.5}, wherein eve more preferably,
at least one of the one or more solvents capable of dissolving the hydrophobic polymer of (a) are selected from dioctylcyclohexane, mineral oil, isocetyl palmitate, isocetyl palmitate, cyclopentasiloxane, dicaprylyl carbonate, octyl isostearate, trimethylhexyl isononanoate, 2-ethylhexyl isononanoate, dicaprylyl ether, dihexyl carbonate, polydecene, octyl cocoate, isodecyl neopentanoate, isohexy decanoate, isodecyl octanoate, dihexyl ether, isododecane, isodecyl 3,5,5 trimethyl hexanoate, oleyl erucate, Passiflora incarnata oil, jojoba oil, octyl palmitate, macadamia nut oil, isopropyl stearate, rapeseed oil, hexyl decanol, isotridecyl 3,5,5 trimethylhexanonanoate, polycitronellol acetate, mixed decanoyl and octanoyl glycerides, 2-ethylhexanoic acid, 3,5,5 trimethyl ester, cetystearyl octanoate, dimethicone, isopropyl palmitate, octyldodecanol, dioctyl adipate, isopropyl myristate, octyl palmitate (2-ethylhexyl palmitate), octyldodeceyl myristate, butyl octanoic acid, isopropyl stearate, caprylic/capric triglycerides, isopropyl isostearate, Jojoba oil, cyclomethicone, groundnut oil, almond oil, sunflower oil, decyl oleate, avocado oil, olive oil, dibutyl adipate, castor oil, calendula oil, wheatgerm oil, decyl oleate, avocado oil, calendula oil, propylene glycol monoisostearate, cocoglycerides, butylene glycol caprylate/caprate, C12-15 alkyl benzoate, caprylic/capric diglyceryl succinate, caprylic/capric triglyceride, cetearyl isonoanoate, cetearyl octanoate, cetyl dimethicone, coco-caprylate/caprate, cocoglycerides, Di-C12-13 alkyl tartrate, dibutyl adipate, dicaprylyl carbonate, dicaprylyl ether, hexyl decanol, hydrogenated polyisobutene, isoeicosane, isohexadecane, isopropyl palmitate, isopropyl stearate, octyl cocoate, octyl isostearate, octyl octanoate, octyl palmitate, octyl stearate, octyl dodecanol, octyldodecyl myristate, isopropyl stearate, pentaerythrityl tetraisostearate, phenyl trimethicone, polydecene, propylene glycol dicaprylate/dicaprate, stearyl heptanoate, tricaprylin, tridecyl stearate, tridecyl trimellitate, triisostearin, or combinations thereof;
(c) one or more skin active agents, wherein preferably the one or more skin active agents include a ceramide;
(d) about 0.1 to about 15 wt. %, preferably about 0.5 to about 10 wt. %, more preferably about 1 to about 8 wt. % of one or more surfactants, preferably a plurality of surfactants; and
(e) water;
(f) optionally, one or more miscellaneous ingredients, preferably wherein the one or more miscellaneous ingredients, if present, are selected from preservatives, fragrances, pH adjusters, salts, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, wherein if present, are preferably in an amount of about 0.1 to about 15 wt. %, more preferably about 0.5 to about 10 wt. %, more preferably about 1 to about 6 wt. %;
   wherein the composition is an oil-in-water emulsion.

The average droplet size of the droplets in the dispersion is preferably from about 10 nm to about 1 μm, about 10 nm to about 800 nm, about 10 nm to about 600 nm, about 10 nm to about 500 nm, about 10 nm to about 250 nm, about 50 nm to about 1 μm, about 50 nm to about 800 nm, about 50 nm to about 600 nm, about 50 nm to about 500 nm, about 50 nm to about 250 nm, about 100 nm to about 1 μm, about 100 nm to about 800 nm, about 100 nm to about 600 nm, about 100 nm to about 500 nm, about 100 nm to about 300 nm, about 150 nm to about 500 nm, about 150 nm to about 400 nm, about 200 nm to about 500 nm, or about 200 nm to about 300 nm.

The pH of the cosmetic composition may be from about 4.5 to about 8.5. In further embodiments, the pH of the emulsion may be from about 5 to about 8, about 5 to about 7, about 5 to less than 7, about 4.5 to about 6.5, about 5 to about 6.5, about 5.5 to about 6.5, about 5 to about 6, or about 6 to about 7.5.

In preferred embodiments, the cosmetic composition comprises, consists of, or consists essentially of:
(a) about 0.1 to about 8 wt. %, preferably about 0.5 to about 5 wt. %, more preferably about 1 to about 5 wt. % of a hydrophobic polymer, which is a reaction product of a natural or food-derived oil and a methacrylate or acrylate polymer, wherein preferably,
   the hydrophobic polymer is a reaction product of: (a)(i) a natural or food-derived oil selected from linseed oil, sunflower oil, tung oil, fish oil, cottonseed oil, soybean oil, or combinations thereof, preferably linseed oil and (a)(ii) a polymer derived from monomers selected from isobutyl methacrylate, methyl methacrylate, ethyl methacrylate, n-butyl methacrylate, and combinations thereof, preferably isobutyl methacrylate polymer, wherein even more preferably,
   the hydrophobic polymer is the reaction product of linseed oil and poly(isobutyl methacrylate);
(b) about 0.1 to about 25 wt. %, preferably about 0.5 to about 15 wt. %, more preferably about 0.5 to about 5 wt. % of one or more solvents capable of solubilizing the reaction product of (a), wherein preferably,
   the one or more solvents capable of solubilizing the reaction product of (a) have a dispersion component (D), a polar component (P), and a hydrogen bonding component (H), and a distance (Ra) of less than or equal to 13.4 MPa^{0.5} per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
D₁ is 16.8 MPa^{0.5},
P₁ is 4.8 MPa^{0.5}, and
H₁ is 13.0 MPa^{0.5}, wherein more preferably,
the one or more solvents capable of solubilizing the reaction product of (a) have a dispersion component (D), a polar component (P), and a hydrogen bonding component (H), and a distance (Ra) of less than or equal to 9.9 MPa^{0.5} per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
D₁ is 16.4 MPa^{0.5},
P₁ is 5.0 MPa^{0.5}, and
H₁ is 11.7 MPa^{0.5}, wherein eve more preferably,
at least one of the one or more solvents capable of dissolving the hydrophobic polymer of (a) are selected from dioctylcyclohexane, mineral oil, isocetyl palmitate, isocetyl palmitate, cyclopentasiloxane, dicaprylyl carbonate, octyl isostearate, trimethylhexyl isononanoate, 2-ethylhexyl isononanoate, dicaprylyl ether, dihexyl carbonate, polydecene, octyl cocoate, isodecyl neopentanoate, isohexy decanoate, isodecyl octanoate, dihexyl ether, isododecane, isodecyl 3,5,5 trimethyl hexanoate, oleyl erucate, Passiflora incarnata oil, jojoba oil, octyl palmitate, macadamia nut oil, isopropyl stearate, rapeseed oil, hexyl decanol, isotridecyl 3,5,5 trimethylhexanonanoate, polycitronellol acetate, mixed decanoyl and octanoyl glycerides, 2-ethylhexanoic acid, 3,5,5 trimethyl ester, cetystearyl octanoate, dimethicone, isopropyl palmitate, octyldodecanol, dioctyl adipate, isopropyl myristate, octyl palmitate (2-ethylhexyl palmitate), octyldodeceyl myristate, butyl octanoic acid, isopropyl stearate, caprylic/capric triglycerides, isopropyl isostearate, Jojoba oil, cyclomethicone, groundnut oil, almond oil, sunflower oil, decyl oleate, avocado oil, olive oil, dibutyl adipate, castor oil, calendula oil, wheatgerm oil, decyl oleate, avocado oil, calendula oil, propylene glycol monoisostearate, cocoglycerides, butylene glycol caprylate/caprate, C12-15 alkyl benzoate, caprylic/capric diglyceryl succinate, caprylic/capric triglyceride, cetearyl isonoanoate, cetearyl octanoate, cetyl dimethicone, coco-caprylate/caprate, cocoglycerides, Di-C12-13 alkyl tartrate, dibutyl adipate, dicaprylyl carbonate, dicaprylyl ether, hexyl decanol, hydrogenated polyisobutene, isoeicosane, isohexadecane, isopropyl palmitate, isopropyl stearate, octyl cocoate, octyl isostearate, octyl octanoate, octyl palmitate, octyl stearate, octyl dodecanol, octyldodecyl myristate, isopropyl stearate, pentaerythrityl tetraisostearate, phenyl trimethicone, polydecene, propylene glycol dicaprylate/dicaprate, stearyl heptanoate, tricaprylin, tridecyl stearate, tridecyl trimellitate, triisostearin, or combinations thereof;
(c) about 0.01 to about 10 wt. %, preferably about 0.1 to about 8 wt. %, more preferably about 0.5 to about 5 wt. % of one or more skin active agents; wherein preferably at least one of the one or more skin active agents is a ceramide selected from Ceramide EOP, Ceramide AS, Ceramide AP, Ceramide NS, Ceramide NP, Ceramide NH, Ceramide AH, Ceramide EOH, Ceramide EOS, Ceramide AdS, Ceramide NdS, Ceramide EOdS, or mixtures thereof;
(d) about 0.1 to about 15 wt. %, preferably about 0.5 to about 10 wt. %, more preferably about 1 to about 8 wt. % of one or more surfactants, wherein the surfactants are selected from:
   (d)(i) one or more biosurfactants, preferably wherein at least one of the one or more biosurfactants is a glycolipid selected from sophorolipids, rhamnolipids, trehalose lipids, mannosylerythritol lipids, and combinations thereof, wherein preferably at least one of the one or more biosurfactants is a rhamnolipid; and/or
   (d)(ii) one or more additional surfactants other than the one or more biosurfactants of (d)(i), preferably wherein the one or more additional surfactants other than the biosurfactants of (d)(i) include one or more anionic surfactants, one or more nonionic surfactants, one or more amphoteric surfactants, or combination thereof;
(e) about 75 to about 95 wt. %, preferably about 80 to about 95 wt. %, more preferably about 85 to about 93 wt. % of water;
(r) optionally, one or more water soluble solvents, preferably wherein the one or more water soluble solvents, if present, is selected from mono-alcohols (for example C₂₋₈, or C₂₋₄ alcohols), polyols (polyhydric alcohols), glycols, and a mixture thereof, wherein if present, the one or more water soluble solvents is in an amount of about 0.01 to about 20 wt. %, preferably about 0.1 to about 15 wt. %, more preferably about 0.5 to about 10 wt. %; and
(g) optionally, one or more film forming polymers; and
(h) optionally, one or more miscellaneous ingredients, preferably wherein the one or more miscellaneous ingredients, if present, are selected from preservatives, fragrances, pH adjusters, salts, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, wherein if present, are preferably in an amount of about 0.1 to about 15 wt. %, more preferably about 0.5 to about 10 wt. %, more preferably about 1 to about 6 wt. %;
   wherein the composition is an oil-in-water emulsion, and
   all percentages by weight are based on the total weight of the composition.

The average droplet size of the droplets is preferably from about 10 nm to about 1 μm, about 10 nm to about 800 nm, about 10 nm to about 600 nm, about 10 nm to about 500 nm, about 10 nm to about 250 nm, about 50 nm to about 1 μm, about 50 nm to about 800 nm, about 50 nm to about 600 nm, about 50 nm to about 500 nm, about 50 nm to about 250 nm, about 100 nm to about 1 μm, about 100 nm to about 800 nm, about 100 nm to about 600 nm, about 100 nm to about 500 nm, about 100 nm to about 300 nm, about 150 nm to about 500 nm, about 150 nm to about 400 nm, about 200 nm to about 500 nm, or about 200 nm to about 300 nm.

The pH of the cosmetic composition may be from about 4.5 to about 8.5. In further embodiments, the pH of the emulsion may be from about 5 to about 8, about 5 to about 7, about 5 to less than 7, about 4.5 to about 6.5, about 5 to about 6.5, about 5.5 to about 6.5, about 5 to about 6, or about 6 to about 7.5.

In another preferred embodiment, the cosmetic composition comprises, consists essentially of, or consists of:
(a) about 0.1 to about 8 wt. %, preferably about 0.5 to about 5 wt. %, more preferably about 1 to about 5 wt. % of a hydrophobic polymer which is the reaction product of linseed oil and poly(isobutyl methacrylate);
(b) about 5 to about 60 wt. %, preferably about 10 to about 50 wt. %, more preferably about 20 to about 45 wt. % of one or more solvents capable of solubilizing the reaction product of (a), wherein preferably,
   the one or more solvents capable of solubilizing the reaction product of (a) have a dispersion component (D), a polar component (P), and a hydrogen bonding component (H), and a distance (Ra) of less than or equal to 13.4 MPa^{0.5} per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
D₁ is 16.8 MPa^{0.5},
P₁ is 4.8 MPa^{0.5}, and
H₁ is 13.0 MPa^{0.5}, wherein more preferably,
the one or more solvents capable of solubilizing the reaction product of (a) have a dispersion component (D), a polar component (P), and a hydrogen bonding component (H), and a distance (Ra) of less than or equal to 9.9 MPa^{0.5} per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
D₁ is 16.4 MPa^{0.5},
P₁ is 5.0 MPa^{0.5}, and
H₁ is 11.7 MPa^{0.5}, wherein eve more preferably,
at least one of the one or more solvents capable of dissolving the hydrophobic polymer of (a) are selected from dioctylcyclohexane, mineral oil, isocetyl palmitate, isocetyl palmitate, cyclopentasiloxane, dicaprylyl carbonate, octyl isostearate, trimethylhexyl isononanoate, 2-ethylhexyl isononanoate, dicaprylyl ether, dihexyl carbonate, polydecene, octyl cocoate, isodecyl neopentanoate, isohexy decanoate, isodecyl octanoate, dihexyl ether, isododecane, isodecyl 3,5,5 trimethyl hexanoate, oleyl erucate, Passiflora incarnata oil, jojoba oil, octyl palmitate, macadamia nut oil, isopropyl stearate, rapeseed oil, hexyl decanol, isotridecyl 3,5,5 trimethylhexanonanoate, polycitronellol acetate, mixed decanoyl and octanoyl glycerides, 2-ethylhexanoic acid, 3,5,5 trimethyl ester, cetystearyl octanoate, dimethicone, isopropyl palmitate, octyldodecanol, dioctyl adipate, isopropyl myristate, octyl palmitate (2-ethylhexyl palmitate), octyldodeceyl myristate, butyl octanoic acid, isopropyl stearate, caprylic/capric triglycerides, isopropyl isostearate, Jojoba oil, cyclomethicone, groundnut oil, almond oil, sunflower oil, decyl oleate, avocado oil, olive oil, dibutyl adipate, castor oil, calendula oil, wheatgerm oil, decyl oleate, avocado oil, calendula oil, propylene glycol monoisostearate, cocoglycerides, butylene glycol caprylate/caprate, C12-15 alkyl benzoate, caprylic/capric diglyceryl succinate, caprylic/capric triglyceride, cetearyl isonoanoate, cetearyl octanoate, cetyl dimethicone, coco-caprylate/caprate, cocoglycerides, Di-C12-13 alkyl tartrate, dibutyl adipate, dicaprylyl carbonate, dicaprylyl ether, hexyl decanol, hydrogenated polyisobutene, isoeicosane, isohexadecane, isopropyl palmitate, isopropyl stearate, octyl cocoate, octyl isostearate, octyl octanoate, octyl palmitate, octyl stearate, octyl dodecanol, octyldodecyl myristate, isopropyl stearate, pentaerythrityl tetraisostearate, phenyl trimethicone, polydecene, propylene glycol dicaprylate/dicaprate, stearyl heptanoate, tricaprylin, tridecyl stearate, tridecyl trimellitate, triisostearin, or combinations thereof;
(c) about 0.01 to about 10 wt. %, preferably about 0.1 to about 8 wt. %, more preferably about 0.5 to about 5 wt. % of one or more skin active agents; wherein preferably at least one of the one or more skin active agents is a ceramide selected from Ceramide EOP, Ceramide AS, Ceramide AP, Ceramide NS, Ceramide NP, Ceramide NH, Ceramide AH, Ceramide EOH, Ceramide EOS, Ceramide AdS, Ceramide NdS, Ceramide EOdS, or mixtures thereof;
(d) about 0.1 to about 15 wt. %, preferably about 0.5 to about 10 wt. %, more preferably about 1 to about 8 wt. % of one or more surfactants, preferably a plurality of surfactants, wherein the plurality of surfactants are preferably selected from
   (d)(i) one or more biosurfactants, preferably wherein at least one of the one or more biosurfactants is a glycolipid selected from sophorolipids, rhamnolipids, trehalose lipids, mannosylerythritol lipids, and combinations thereof, wherein preferably at least one of the one or more biosurfactants is a rhamnolipid; and/or
   (d)(ii) one or more additional surfactants other than the one or more biosurfactants of (d)(i), preferably wherein the one or more additional surfactants other than the biosurfactants of (d)(i) include one or more anionic surfactants, one or more nonionic surfactants, one or more amphoteric surfactants, or combination thereof;
(e) about 30 to about 90 wt. %, preferably about 40 to about 80 wt. %, more preferably about 40 to about 70 wt. % of water;
(f) optionally, one or more miscellaneous ingredients, preferably wherein the one or more miscellaneous ingredients, if present, are selected from preservatives, fragrances, pH adjusters, salts, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, wherein if present, are preferably in an amount of about 0.1 to about 15 wt. %, more preferably about 0.5 to about 10 wt. %, more preferably about 1 to about 6 wt. %;
   wherein the composition is an oil-in-water emulsion.

The average droplet size of the droplets in the dispersion is preferably from about 10 nm to about 1 μm, about 10 nm to about 800 nm, about 10 nm to about 600 nm, about 10 nm to about 500 nm, about 10 nm to about 250 nm, about 50 nm to about 1 μm, about 50 nm to about 800 nm, about 50 nm to about 600 nm, about 50 nm to about 500 nm, about 50 nm to about 250 nm, about 100 nm to about 1 μm, about 100 nm to about 800 nm, about 100 nm to about 600 nm, about 100 nm to about 500 nm, about 100 nm to about 300 nm, about 150 nm to about 500 nm, about 150 nm to about 400 nm, about 200 nm to about 500 nm, or about 200 nm to about 300 nm.

The pH of the cosmetic composition may be from about 4.5 to about 8.5. In further embodiments, the pH of the emulsion may be from about 5 to about 8, about 5 to about 7, about 5 to less than 7, about 4.5 to about 6.5, about 5 to about 6.5, about 5.5 to about 6.5, about 5 to about 6, or about 6 to about 7.5.

### EXAMPLES

Implementation of the present disclosure is provided by way of the following examples. The examples serve to illustrate the technology without being limiting in nature.

### Example 1

Composition A is an inventive composition containing MycelX®. Composition B is identical to composition A but does not include the MycelX®. Inventive composition A was manufactured by first combining the MycelX® (composition A), the caprylic/capric triglyceride, surfactants (mRhamnolipid and sodium methyl cocoyl taurate), and ceramide, with only a small amount of water (about 15 to about 30 wt. %, based on the total weight of the final cosmetic composition). A homogenous composition was obtained, which was further diluted with water and gently mixed or shaken to form the final oil-in-water emulsion. Comparative composition B was prepared based on a standard preparation procedure for oil-in-water emulsions. The ingredients were combined, without generating a first composition with a limited amount of water. The composition was processed with a high-speed Silverson Homogenizer capable of generating fine droplet sizes in the range of 2-5 microns. Despite subjecting the compositions to the high-speed homogenization process, the droplet size of the resulting emulsions was greater than 1 micron.

| | | Inventive | Comparative |
|---|---|---|---|
| | | A | B |
| (a) | MYCELX ®¹ | 0.5 | |
| (b) | CAPRYLIC/CAPRIC TRIGLYCERIDE | 0.3 | 0.3 |
| (c) | m-RHAMNOLIPID | 0.6 | 0.6 |
| | SODIUM METHYL COCOYL TAURATE | 0.2 | 0.2 |
| (d) | Ceramides² | 1 | 1 |
| (e) | Water | QS | QS |
| DROPLETS SIZE BY DILUTION | | ~150 nm | > 4 μm |
| 1:100 [nm] | | | |
| Stable 45° C. 2 Months | | Yes | No |
| Young's modulus Ecore by AFM (Mpa) | | ~60-300 | NA |

¹Reaction product of linseed oil and isobutyl methacrylate polymer
²Blend of Ceramide-EOP, Ceramide-NP, and Ceramide-EP

After forming the emulsions, the compositions were further diluted by a 1:100 ratio with water and the droplet size measured using Brookhaven Dynamic Light Scattering (DLS). DLS is a technique used to determine droplet size in a colloidal system or emulsion. A Brookhaven Instrument was used to determine the size distribution of particles in each sample. When the samples are illuminated with a monochromatic laser beam, the particles in the samples undergo Brownian motion, causing fluctuations in scattered light intensity. The scattered light is then collected at various angles, and the autocorrelation function of these intensity fluctuations was analyzed. The analysis provides information about the rate of diffusion of the particles, and from this, the size distribution is inferred using mathematical models. Brookhaven's DLS instruments use advanced algorithms to accurately interpret the data, offering insights into the dynamic behavior and size characteristics of particles ranging from a few nanometers to several micrometers in a liquid medium.

Cryo-Scanning Electron Microscopy (Cryo-SEM) was used to visualize the spherical droplets/structures formed in inventive composition A. Cryo-SEM is a technique used to examine the ultrastructure of biological and other samples at cryogenic temperatures. It involves rapidly freezing the sample to preserve its native structure, transferring it to the SEM chamber while maintaining cryogenic conditions, and then imaging the sample's surface using electrons. This method provides high-resolution images of samples, minimizing beam damage and charging effects, making it suitable for various applications, including structural analysis, cellular imaging, and the study of soft materials. An image of a spherical droplet/structures formed in inventive composition A is shown in The FIGURE.

Atomic Force Microscopy (AFM) is a high-resolution imaging and surface characterization technique used in nanoscience, materials science, and biology. AFM operates by scanning a sharp tip (typically a few nanometers in diameter) attached to a flexible cantilever very close to the sample surface. The interaction forces between the tip and the sample surface cause the cantilever to deflect, and this deflection is measured to create high-resolution images and gather information about surface properties. AFM can provide topographical information at the nanoscale, measure mechanical properties, and even manipulate individual atoms and molecules on a surface. The spherical droplet/structures formed in inventive composition A were evaluated by AFM. They were smaller (<1 micron) and more uniformly dispersed (monodispersed) than the droplets of comparative composition B and they exhibited a soft shell and hard core with Young's modulus Ecore ˜60 −300 Mpa by AFM.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

As used herein, the terms “comprising,” “having,” and “including” are used in their open, non-limiting sense.

The terms “a,” “an,” and “the” are understood to encompass the plural as well as the singular. Thus, the term “a mixture thereof” also relates to “mixtures thereof.” Throughout the disclosure, the term “a mixture thereof” is used, following a list of elements as shown in the following example where letters A-F represent the elements: “one or more elements selected from the group consisting of A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counterion. This list of counterions, however, is non-limiting. Appropriate counterions for the components described herein are known in the art.

The expression “one or more” means “at least one” and thus includes individual components as well as mixtures/combinations.

The term “plurality” means “more than one” or “two or more.”

The term “transparent” with respect to a transparent composition indicates that the composition has transmittance of at least 80% at a wavelength of 600 nm, for example measured using a Lambda 40 UV-visible spectrometer. The compositions may have, for example, a transmittance of at least 80%, at least 90%, or at least 95% at a wavelength of 600 nm, measured, for example, using a Lambda 40 UV-visible spectrometer. The term “clear” is interchangeable with the term “transparent” for purposes of the instant disclosure. In various embodiments, the cosmetic compositions of the instant disclosure are transparent. This is due, at least in part, to the small droplet size of the dispersed oil droplets.

The term “translucent” with respect to a translucent composition indicates that the composition has a transmittance of at least 50% at a wavelength of 600 nm, for example measured using a Lambda 40 UV-visible spectrometer. In various embodiments, the cosmetic compositions of the instant disclosure are translucent. This is due, at least in part, to the small droplet size of the dispersed oil droplets.

Other than in the operating examples, or where otherwise indicated, all amount expressing quantities of ingredients and/or reaction conditions may be modified in all instances by the term “about,” meaning within +/−5% of the indicated number. Thus, for a range of “about 1 to about 10 wt. %,” The lower amount of “about 1 wt. %” may extend down to 0.95 wt. %, which is 5% less than 1 wt. %. The higher amount of “about 10 wt. %” may extend up to 10.5 wt. %, which is 5% higher than 10 wt. %, i.e., a range of “0.95 wt. % to 10.5 wt. %.”

All percentages, parts and ratios herein are based upon the total weight of the compositions of the present invention, unless otherwise indicated.

Some of the various categories of components identified may overlap. In such cases where overlap may exist and the composition includes both components (or the composition includes more than two components that overlap), an overlapping compound does not represent more than one component. For example, certain compounds may be considered both oily solvent and a surfactant. If a particular composition includes both an oily solvent and a surfactant, a single compound will serve as only the oily solvent or only as the surfactant (the single compound does not simultaneously serve as both the oily solvent and the surfactant).

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

The term “substantially free” or “essentially free” as used herein means that there is less than about 2% by weight of a specific material added to a composition, based on the total weight of the compositions. Nonetheless, the compositions may include less than about 1 wt. %, less than about 0.5 wt. %, less than about 0.1 wt. %, or none of the specified material. For example, if a composition is essentially free from compound X, the composition includes less that 2 wt. % of compound X, or less than 1 wt. % of compound X, or less than 0.5 wt. % of compound X, or less than 0.1 wt. % of compound X, or is free from compound X.

All components that are positively set forth in the instant disclosure may be negatively excluded from the claims, e.g., a claimed composition may be “free,” “essentially free” (or “substantially free”) of one or more components that are positively set forth in the instant disclosure.

All publications and patent applications cited in this specification are herein incorporated by reference in their entirety, and for any and all purposes, as if each individual publication or patent application were specifically and individually indicated to be incorporated by reference. In the event of an inconsistency between the present disclosure and any publications or patent application incorporated herein by reference, the present disclosure controls.

1. A cosmetic composition comprising:
(a) hydrophobic polymer formed as a reaction product of a natural or food-derived oil and a methacrylate or acrylate polymer;
(b) one or more solvents capable of solubilizing the reaction product of (a);
(c) one or more skin active agents;
(d) one or more surfactants; and
(e) water;
wherein the composition is an oil-in-water emulsion.

2. The composition of claim 1, wherein the hydrophobic polymer is the reaction product formed from a natural or food-derived oil and a methacrylate polymer.

3. The composition of claim 1, wherein the natural or food-derived oil is a drying oil or semi-drying oil selected from linseed oil, sunflower oil, tung oil, fish oil, cottonseed oil, soybean oil, or combinations thereof.

4. The composition of claim 1, wherein the polymer is derived from monomers selected from isobutyl methacrylate, methyl methacrylate, ethyl methacrylate, n-butyl methacrylate, and combinations thereof.

5. The composition of claim 1, wherein the hydrophobic polymer is the reaction product of about 50 to about 85 parts by weight of the natural or food-derived oil and about 15 to about 50 parts by weight of the methacrylate or acrylate polymer.

6. The composition of claim 1, wherein the hydrophobic polymer is the reaction product of about 72 to about 77 parts by weight of the natural or food-derived oil and about 23 to about 28 parts by weight of a methacrylate polymer.

7. The composition of claim 1, wherein the polymer is isobutyl methacrylate polymer.

8. The composition of claim 1, wherein the hydrophobic polymer is the reaction product of linseed oil and poly(isobutyl methacrylate).

9. The composition of claim 1, wherein the one or more solvents capable of solubilizing the reaction product of (a) have a dispersion component (D), a polar component (P), a hydrogen bonding component (H), and a distance (Ra) of less than or equal to 13.4 MPa^{0.5} per Hansen Solubility Parameters, wherein the distance (Ra) is defined by formula (I):
$$Ra = \sqrt{4 {( D - {D}_{1} )}^{2} + {( P - {P}_{1} )}^{2} + {( H - {H}_{1} )}^{2}} \qquad ( I )$$
wherein
D₁ is 16.8 MPa^{0.5},
P₁ is 4.8 MPa^{0.5}, and
H₁ is 13.0 MPa^{0.5}.

10. The composition of claim 1, wherein at least one of the one or more solvents capable of solubilizing the hydrophobic polymer of (a) is selected from polycitronellol acetate, caprylic/capric triglyceride, isododecane, isohexadecane, tetradecane, isopropyl myristate, isopropyl alcohol, octyldodecanol, ethanol, phenoxyethanol, castor oil, or mixtures thereof.

11. The composition of claim 1, wherein the one or more skin active agents are selected from ceramides, vitamin A, vitamin C, vitamin D, vitamin E, niacinamide, caffeine, ferulic acid, salicylic acid, madecassoside, retinoic acid, benzoyl peroxide, or a combinations thereof.

12. The composition of claim 1, wherein the one or more skin active agents include at least one ceramide selected from Ceramide EOP, Ceramide AS, Ceramide AP, Ceramide NS, Ceramide NP, Ceramide NH, Ceramide AH, Ceramide EOH, Ceramide EOS, Ceramide AdS, Ceramide NdS, Ceramide EOdS, or mixtures thereof.

13. The cosmetic composition of claim 1, wherein the one or more skin active agents is a lipophilic skin active agent.

14. The composition of claim 1, wherein the one or more biosurfactants are selected from glycolipids, wherein the biosurfactants are selected from sophorolipids, rhamnolipids, trehalose lipids, mannosylerythritol lipids, or mixtures thereof.

15. The composition of claim 1, wherein the one or more surfactants are selected from anionic surfactants, nonionic surfactants, amphoteric surfactants, or mixtures thereof.

16. The composition of claim 15 comprising one or more anionic surfactants, wherein the one or more anionic surfactants are selected from acyl glutamates, acyl taurates, alkanoyl isethionates, alkyl succinates, alkyl sulphosuccinates, N-alkoyl sarcosinates, alkyl phosphates, alkyl ether phosphates, alkyl ether carboxylates, alpha-olefin sulphonates, or mixtures thereof.

17. The composition of claim 16, wherein at least one of the one or more anionic surfactants is an acyl taurate.

18. The composition of claim 1, wherein the oil-in-water emulsion is a dispersion having a droplet size from about 10 nm to about 1 μm.

19. A cosmetic composition comprising:
(a) about 0.5 to about 10 wt. % of a reaction product of a natural or food-derived oil selected from linseed oil, sunflower oil, tung oil, fish oil, cottonseed oil, soybean oil, or mixtures thereof, and a polymer formed from one or more monomers selected from isobutyl methacrylate, methyl methacrylate, ethyl methacrylate, and n-butyl methacrylate or mixtures thereof,
(b) about 0.1 to about 10 wt. % of one or more solvents capable of solubilizing the reaction product of (a);
(c) one or more ceramides;
(d) about 1 to about 10 wt. % of a plurality of surfactants, wherein the plurality of surfactants comprise:
(c)(i) about 0.1 to about 8 wt. % of one or more rhamnolipids; and
(c)(ii) about 0.1 to about 8 wt. % of one or more additional surfactants selected from anionic surfactants, nonionic surfactants, amphoteric surfactants, and cationic surfactants; and
(d) water;
wherein the composition has an average droplet size of about 10 nm to about 2 μm, and
all percentages by weight are based on a total weight of the composition.

20. A method for treating skin comprising applying the cosmetic composition of claim 1 to the skin.
